(19) United States
(12) Patent Application Publication
(10) Pub. No.: US 2025/0320534 A1
(43) Pub. Date: Oct. 16, 2025

(54) COMPOSITIONS AND METHODS FOR PRODUCING DIHYDROFURANS FROM KETO-SUGARS

(71) Applicants: Arzeda Corp., Seattle, WA (US); BP Corporation North America Inc., Houston, TX (US)

(72) Inventors: Amandeep SANGHA, Seattle, WA (US); Karen EATON, Seattle, WA (US); James LEINAS, Seattle, WA (US); Eric Anthony ALTHOFF, Seattle, WA (US); Lu YANG, Houston, TX (US); Christopher M. PHILLIPS, Houston, TX (US); Liang SONG, Houston, TX (US); Yongmei HU, Houston, TX (US)

(21) Appl. No.: 18/957,943

(22) Filed: Nov. 25, 2024

Related U.S. Application Data
(63) Continuation of application No. PCT/US2023/068421, filed on Jun. 14, 2023.
(60) Provisional application No. 63/352,145, filed on Jun. 14, 2022.

(51) Int. Cl. C12P 17/04; C07D 307/32; C07D 307/68; C12N 1/20; C12N 9/24; C12R 1/19

(52) U.S. Cl. CPC C12P 17/04; C07D 307/32; C07D 307/68; C12N 1/20; C12N 9/2402; C12R 2001/19

(57) ABSTRACT

Provided are compositions and methods for producing dihydrofurans by way of glycosyl hydrolases that can dehydrate 2-keto-3-deoxy-gluconate (KDG) to K4. Provided are also compositions and methods for further processing K4 to create HMFA (5-hydroxymethyl-2-furoic acid) and/or FDCA (2,5-furan dicarboxylic acid).

### CROSS-REFERENCE TO RELATED APPLICATIONS

This application is a continuation of International Patent Application No. PCT/US2023/068421, filed Jun. 14, 2023. which claims the benefit of U.S. Provisional Patent Application No. 63/352,145, filed on Jun. 14, 2022, the content of each of which is herein incorporated by reference in its entirety.

### REFERENCE TO AN ELECTRONIC SEQUENCE LISTING

The contents of the electronic sequence listing (ARZE_035_03US_SeqList_ST26.xml; Size: 144,113 bytes: and Date of Creation: November 13. 2024) are herein incorporated by reference in its entirety.

### BACKGROUND

Furandicarboxylic acid (FDCA) is one of the main building blocks of polyethylene 2,5-furandicarboxylate (PEF), which is a plant-based polymer that has 50-70% less carbon footprint than its petroleum-based competitor polyethylene terephthalate (PET). Currently PET dominates the world market for packaging because it is strong, lightweight, and does not shatter. The majority of PET is used for creation of synthetic fiber and the remaining 30% is used for bottle production. However, PEF has multiple advantages over PET, in that it can be reused up to 5× more times when combined with PET than when using PET alone, it degrades much faster than PET, and it can be substituted for film packing that is currently not recyclable.

Previous methods to produce FDCA or produce even more basic and natural carbohydrate starting materials such as dihydrofurans have focused on chemical-catalysis methods in industry. However, these methods involve utilizing high-cost catalysts, which also exhibit significant constraints and process drawbacks. Currently there are no industry adopted biocatalytic processes that can produce FDCA yields comparable to the chemical-catalytic routes.

Thus, there is a need to provide novel enzymes and methods to produce dihydrofurans from keto-sugars using biocatalytic routes in thermochemically favorable conditions.

### SUMMARY

Provided herein are methods for generating a dihydrofuran from keto-sugars.

In an embodiment, the present disclosure relates to a biocatalytic method of generating a dihydrofuran, the method comprising contacting a 2-keto-3-deoxygluconate (KDG) with a glycoside hydrolase, thereby generating the dihydrofuran, wherein the contacting comprises: a. a pH from about 4 to about 7 as determined by pH meter: b. a temperature from 45° C. to 74° C.: or c. both a. and b., thereby generating the dihydrofuran. In an embodiment, the method comprises a., wherein the pH is from about 4 to 5. In an embodiment, the method comprises b., wherein the temperature is from 70° C. to 74° C. In an embodiment, the method comprises a. and b., wherein the pH is from about 4 to 5 and the temperature is from about 62° C. to 72° C. In an embodiment, the method comprises a. and b., wherein the pH and the temperature are selected from the group consisting of: a pH about 4 and temperature about 63° C.: b pH about 4.5 and temperature about 69° C.: and c. pH about 5 and temperature about 72° C. In an embodiment, the method comprises c. In an embodiment, the KDG is from 180 mM to 300 mM. In an embodiment, the KDG is from 180 mM to 220 mM. In an embodiment, the glycoside hydrolase comprises a protein with at least 80% sequence identity to a sequence selected from the group consisting of SEQ ID NO: 1-116. In an embodiment, the sequence identity is at least 85%, 90%, 95%, 98%, 99%, or 100%. In an embodiment, the glycoside hydrolase comprises a first motif that binds the KDG, and a second motif that comprises a catalytic residue, the catalytic residue comprising aspartic acid, the first motif including at least two residues, a first residue comprising arginine and a second residue comprising tryptophan, phenylalanine, or tyrosine, and the glycoside hydrolase is a homolog to SEQ ID NO: 1 or SEQ ID NO: 19 as determined by SWISS-MODEL modeling. In an embodiment. the glycoside hydrolase comprises a protein with 100% sequence identity to SEQ ID NO: 27. In an embodiment, the contacting is from 0.5 hours to 24 hours. In an embodiment, the contacting is from 0.5 hours to 5 hours. In an embodiment, the contacting is about 3 hours. In an embodiment. the method further comprises dehydrating the dihydrofuran to generate 5-hydroxy methyl-2-furoic acid (HMFA), wherein at least 40% yield of the HMFA is observed after the dehydrating. In an embodiment, the dehydrating comprises contacting the dihydrofuran with an acid selected from the group consisting of: formic acid, hydrochloric acid, sulfuric acid, phosphoric acid, nitric acid, hydrobromic acid, and Ci-6 carboxylic acid. In an embodiment, the acid comprises formic acid. In an embodiment, the method further comprises oxidizing the HMFA to generate 2,5-furandicarboxylic acid (FDCA). In an embodiment, the oxidizing comprises a chemical oxidation reaction. In an embodiment, the oxidizing comprises an enzymatic oxidation reaction.

In an embodiment, the present disclosure relates to an isolated polypeptide that comprises at least 85% identity to any one of SEQ ID NO: 35 to SEQ ID NO: 116. In embodiments, the present disclosure further relates to a biocatalytic method of generating a dihydrofuran. the method comprising contacting a 2-keto-3-deoxygluconate (KDG) with the isolated polypeptide to generate the dihydrofuran. In an embodiment, the contacting comprises, a. a pH from about 4 to about 7 as determined by pH meter: b. a temperature from 45° C. to 74° C.: or c. both a. and b., thereby generating the dihydrofuran. In an embodiment, the method comprises a., wherein the pH is from about 4 to 5. In an embodiment, the method comprises b., wherein the temperature is from 70° C. to 74° C. In an embodiment, the method comprises a. and b., wherein the pH is from about 4 to 5 and the temperature is from about 62° C. to 72° C. In an embodiment, the method comprises a. and b., wherein the pH and the temperature are selected from the group consisting of: a. pH about 4 and temperature about 63° C.; b. pH about 4.5 and temperature about 69° C.: and c. pH about 5 and temperature about 72° C. In an embodiment, the method comprises c. In an embodiment, the KDG is from about 180 mM to 300 mM. In an embodiment. the KDG is from about 180 mM to 220 mM. In an embodiment. the contacting is from 0.5 hours to 24 hours. In an embodiment, the contacting is at most 5 hours. In an embodiment, the contacting is about 3 hours. In an embodiment, the method further comprises dehydrating the dihydrofuran to generate 5-hydroxymethyl-2-furoic acid (HMFA). wherein at least 40% yield of the HMFA is observed after the dehydrating. In an embodiment, the dehydrating comprises contacting the dihydrofuran with an acid selected from the group consisting of formic acid, hydrochloric acid, sulfuric acid, phosphoric acid, nitric acid, hydrobromic acid, and Ci-6 carboxylic acid. In an embodiment, the acid comprises formic acid. In an embodiment, the method further comprises oxidizing the HMFA to generate 2,5-furandicarboxylic acid (FDCA). In an embodiment, the oxidizing comprises a chemical oxidation reaction In an embodiment. the oxidizing comprises an enzymatic oxidation reaction. In an embodiment, the present disclosure relates to a composition that comprises the isolated poly peptide.

In an embodiment. the present disclosure relates to a modified microorganism comprising an exogenous glycoside hydrolase, wherein the exogenous glycoside hydrolase comprises a sequence having at least 85% sequence identity to a sequence selected from the group consisting of SEQ ID NOs: 1-116. In an embodiment, the exogenous glycoside hydrolase comprises a sequence having at least 90%, 95%, 97%, 98%, 99%, or 100% sequence identity to the sequence selected from the group consisting of SEQ ID NOs: 1-116. In an embodiment, the exogenous glycoside hydrolase comprises SEQ ID NO: 27. In an embodiment, the modified microorganism is a bacteria. In an embodiment, the bacteria is selected from the group consisting of: E. Coli, Saccharomyces sp., Aspergillus sp., Pichia sp., Pseudomonas sp., and Bacillus sp. In an embodiment, the bacteria is E. Coli. In an embodiment. the present disclosure relates to a composition that comprises the modified microorganism. In an embodiment, the present disclosure relates to a method of generating polyethylene 2,5-furandicarboxylate (PEF), the method comprising the biocatalytic method.

In an embodiment, the present disclosure relates to an isolated unnatural glycoside hydrolase, comprising: a first motif that binds 2-keto-3-deoxygluconate, the first motif including at least two residues, a first residue being arginine and a second residue being tryptophan, phenylalanine, or tyrosine; and a second motif including a catalytic residue, the catalytic residue being aspartic acid or glutamic acid. wherein the isolated unnatural glycoside hydrolase has at least 20% identity to SEQ ID NO: 1 or SEQ ID NO: 19. In an embodiment, the isolated unnatural glycoside hydrolase comprises at least 25%, 45%, 65%, 85%, 95%, or 99% identity to SEQ ID NO: 1 or SEQ ID NO: 19. In an embodiment, the second residue of the at least two residues of the first motif is tryptophan and/or the catalytic residue of the second motif is aspartic acid. In an embodiment, the first motif has a sequence of RxQTW, x being serine or an aliphatic amino acid. In an embodiment, the first motif has a sequence of Rx₁QTW(2x₂)Yx₂Y. x₁ being serine and x₂ being an aliphatic amino acid. In an embodiment, the second motif has a sequence of xD, x being an aliphatic amino acid. In an embodiment, the second motif has a sequence of (2x)KSE(3x)DT(2M)xSxPFx, x being an aliphatic amino acid. In an embodiment, the arginine of the first motif and the catalytic residue of the second motif are separated by about 70 residues.

In an embodiment, the present disclosure relates to an isolated glycoside hydrolase comprising: a sequence of formula 1: P(19x)LPP(4x)HYHQGVxLxG(4x)(W/Y)(10x)Y(3x)(Y/W)x(D/E)(6x)G(9x)D(2x)Q(P/A)Gx (L/I)L(2x)L(7x)(R/K)Y(2x)(A/G)(3x)(L/1)(9x)(T/N)xE(G/Q)G(F/Y)(W/F)H(K/N)(3x)Px(Q/E) (M/Q)WLDGLYMxG(5x)Y(A/G)(9x)D(4x)Q(6x)(H/K)(T/M)(R/K)(3x)TGL(2x)H(A/G)(W/F) (D/S)(2x)(R/K)(3x)W(A/S)(D/N)(2x)(T/S)Gx(S/A)PExW(G/A)R(S/A)xGW(9x)(D/E)x(I/L) P(2x)H(20x)Q(4x)GxWxQ(V/I)x(D/N)(K/R)(G/V)(4x)NW(L/P)ExSx(S/T)xL(6x)K(G/A) (15x)(K/Q)(A/G)(F/Y)xG(18x)(I/V)C(I/V)GT(S/G)xGxY(5x)R(5x)D(L/M)HG(V/A)GA(F/L); and at least 50% homology to SEQ ID NO: 1, wherein x is any amino acid. In an embodiment, the isolated glycoside hydrolase has at least 55%, 60%, 70%, 80%, 90%, 95%, 97%, 98%, 99%, or 100% homology to SEQ ID NO: 1. In an embodiment, the isolated glycoside hydrolase further comprises Y at residue 41; D at residue 88; H at residue 132; W at residue 141; D at residue 143; M at residue 147; H at residue 189; W at residue 211; A at residue 212; R at residue 213; W at residue 217; S at residue 278; L or M at residue 282; C at residue 330; and H or K at residue 352, wherein the residues are numbered according to SEQ ID NO: 1. In an embodiment, the isolated glycoside hydrolase further comprises a loop region having at least 21 residues. In an embodiment, the isolated glycoside hydrolase further comprises a loop region having at least one modification selected from the group consisting of 331 through 336 of SEQ ID NO: 1. In an embodiment, the at least one modification selected from the group consisting of 331 through 336 of SEQ ID NO: 1 includes one or more of V331K, G332E, G332M, G332V, S334A, S334C, S334D, S334E, S334G, S334I, S334K, S334M, S334N, S334Q, S334R, S334T, S334V, A335V, A335P, A335L, and A335C. In an embodiment, the isolated glycoside hydrolase further comprises two amino acids appended at n-terminus of the sequence, and five amino acids appended at the c-terminus of the sequence.

In an embodiment, the present disclosure relates to a biocatalytic method of generating a dihydrofuran, comprising: contacting a 2-keto-3-deoxygluconate (KDG) with a glycoside hydrolase, thereby generating the dihydrofuran, the contacting comprising a. a pH from about 4 to about 7 as determined by pH meter; b. a temperature from 45° C. to 74° C.; or c. both a. and b., thereby generating the dihydrofuran, wherein the glycoside hydrolase has at least 50% homology to SEQ ID NO: 1, and wherein the glycoside hydrolase comprises, relative to SEQ ID NO: 1, D at residue 143, R at residue 213, and W at residue 217. In an embodiment, residue 143 of the glycoside hydrolase is a catalytic residue and residue 213 and residue 217 are substrate binding residues.

In an embodiment, the present disclosure relates to an isolated glycoside hydrolase, comprising: a sequence of formula 2: (F/Y)P(8x)(W/Y)(7x)W(T/M)(2x)F(2x)G(2x)(W/Y)(2x)Y(11x)(A/G)(10x)(L/I)(8x)(H/F)D(L/I) GF(4x)(S/T)(4x)(W/Y)(15x)(A/G)(13x)(L/I)(16x)IDx(L/M)(L/M)(N/S)(22x)H(3x)(T/S)(5x) RxDxS(S/T)(6x)(D/N)(10x)TxQG(4x)SxW(A/S/T)RG(Q/L)(A/T)W(2x)YG(28x)PxD(4x)(Y/W) D(F/L)(12x)S(6x)(S/C)(33x)Y(30x)(W/F/Y)(G/A)DY(Y/F)(2x)ExL; and at least 50% homology to SEQ ID NO: 19, wherein x is any amino acid. In an embodiment, the isolated glycoside hydrolase has at least 55%, 60%, 70%, 80%, 90%, 95%, 97%, 98%, 99%, or 100% homology to SEQ ID NO: 19. In an embodiment, the isolated glycoside hydrolase further comprises I or L at residue 26; H at residue 41; W at residue 42; M at residue 43; H at residue 87; D at residue 88; G at residue 134; D at residue 149; T at residue 150; M at residue 152; Q at residue 193; W at residue 219; R at residue 221; W at residue 225; S at residue 280; I at residue 284; and F at residue 352, wherein the residues are numbered according to SEQ ID NO: 19. In an embodiment, the isolated glycoside hydrolase comprises a sequence selected from the group consisting of SEQ ID NOS: 24 to 27. In an embodiment, the isolated glycoside hydrolase further comprises thirteen amino acids appended at n-terminus of the sequence, and five amino acids appended at the c-terminus of the sequence.

These and other embodiments are described below.

### BRIEF DESCRIPTION OF THE DRAWINGS

The accompanying drawings. which are incorporated herein and form a part of the specification, illustrate some, but not the only or exclusive, example embodiments and/or features. It is intended that the embodiments and drawings disclosed herein are to be considered illustrative rather than limiting.

FIG. 1A shows the conversion (dehydration) of 2-keto-3-deoxy gluconate (KDG) (1) to 4,5-dihydro-4-hydroxy-5-hydroxymethyl-2-furancarboxylic acid (K4) (2) and then further conversion (dehydration) of K4 to 5-hydroxymethyl-2-furoic acid (HMFA) (3).

FIG. 1B is a two-dimensional illustration of KDG.

FIG. 2A shows a representation of SEQ ID NO: 1, a glycoside hydrolase, with KDG bound. The catalytic residues arginine, tryptophan, and aspartic acid are conserved among both Glycosyl hydrolase 88 and 105 family enzymes.

FIG. 2B shows a representative active site of SEQ ID NO: 1, a glycoside hydrolase, with KDG bound. The catalytic residues arginine (1), tryptophan (2), and aspartic acid (3) are conserved among both Glycosyl hydrolase 88 and 105 family enzymes.

FIG. 3 shows an illustration of geometric parameters in the active site of SEQ ID NO: 1 glycosyl hydrolase. Eight geometrical parameters (d indicates distances, θ indicates bond angles) are specified to describe the spatial positions of the functional groups relative to KDG.

FIG. 4 shows K4 detection as determined by an liquid chromatography/mass spectrometry (LC/MS) trace of glycoside hydrolase of SEQ ID NO: 27 that converts KDG to K4 in favorable thermochemical conditions (Na Acetate, pH 4-5, 150 mM NaCl, 63-74° C., 3 hours, 1 uM enzyme, and 180 mM KDG substrate), which ultimately produces HMFA. Glycoside hydrolase, SEQ ID NO: 27, is used to dehydrate KDG to K4, which is then spontaneously and irreversibly dehydrated further to HMFA.

FIG. 5 shows K4 detection as determined by an LC/MS trace of a glycoside hydrolase of SEQ ID NO: 1 that converts KDG to K4 in favorable thermochemical conditions (Na Acetate, pH 5, 25 mM KDG, 45° C., 3 hours), which ultimately produces HMFA. Glycoside hydrolase, SEQ ID NO:1, is used to dehydrate KDG to K4, which is an intermediate that can be detected.

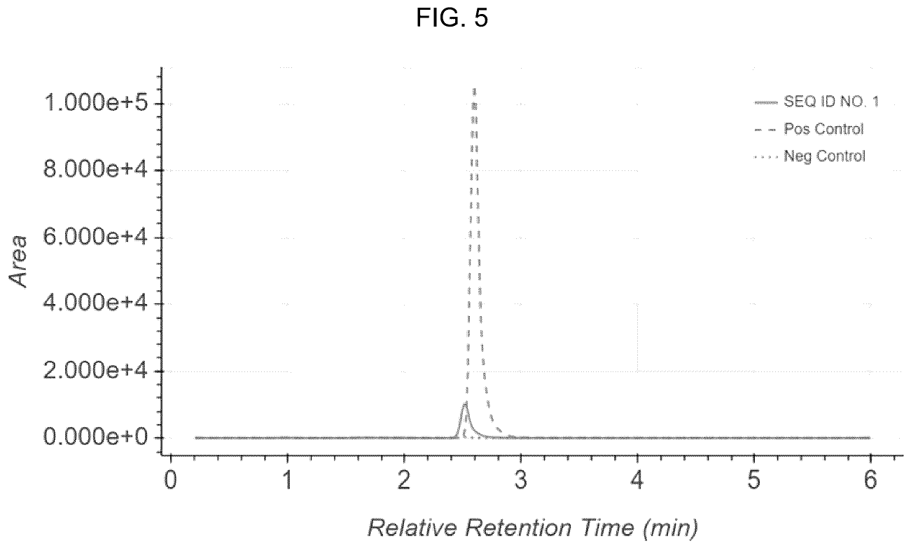

FIG. 6 illustrates an alignment of sequences (SEQ ID Nos: 24 to 27) disclosed herein. The catalytic aspartic acid residue (D) is indicated by “*”, as are the substrate binding residues Arginine (R) and Tryptophan (W).

FIG. 7 illustrates an alignment of key residues for functionality with reference to SEQ ID NO: 19. Corresponding residues from other sequences are provided. Note the high degree of correspondence despite the amino acids being distant in space. For SEQ ID Nos: 23-27, additional residues in common are shown in bold.

### DETAILED DESCRIPTION

The following description includes information that may be useful in understanding the present disclosure. It is not an admission that any of the information provided herein is prior art or relevant to the presently claimed disclosures, or that any publication specifically or implicitly referenced is prior art.

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

As used herein, the singular forms “a,” “an,” and “the: include plural referents unless the content clearly dictates otherwise.

The term “about” or “approximately” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range

As used herein the terms “microorganism” or “microbe” should be taken broadly. These terms are used interchangeably and include, but are not limited to, the two prokaryotic domains, Bacteria and Archaea, as well as certain eukaryotic fungi and protists. In some embodiments, the disclosure refers to the “microorganisms” or “microbes” of lists and figures present in the disclosure. This characterization can refer to not only the identified taxonomic genera but also the identified taxonomic species, as well as the various novel and newly identified or designed strains of any organism in said tables or figures. The same characterization holds true for the recitation of these terms in other parts of the Specification, such as in the Examples.

When referring to a nucleic acid sequence or protein sequence, the term “identity” is used to denote similarity between two sequences Sequence similarity or identity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J Mol. Biol. 48,443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12, 387-395 (1984), or by inspection. Another suitable algorithm is the BLAST algorithm, described in Altschul et al., J Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology. 266, 460-480 (1996); blast.wustl/edu/blast/README.html. WU-BLAST-2 uses several search parameters, which are optionally set to the default values. The parameters are dynamic values and are established by the program itself depending upon the composition of the sequence and composition of the particular database against which the sequence of interest is being searched; however, the values may be adjusted to increase sensitivity. Further, an additional useful algorithm is gapped BLAST as reported by Altschul et al, (1997) Nucleic Acids Res. 25, 3389-3402. Unless otherwise indicated, percent identity is determined herein using the algorithm available at the internet address: blast.ncbi.nlm.nih.gov/Blast.cgi.

As used herein, an “isolated” or “purified” polynucleotide or polypeptide, or biologically active portion thereof, is substantially or essentially free from components that normally accompany or interact with the polynucleotide or poly peptide as found in its naturally occurring environment. Thus, an isolated or purified polynucleotide or polypeptide is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A polypeptide that is substantially free of cellular material includes preparations of polypeptides having less than about 30%, 20%, 10%, 5%, or 1% (by dry weight) of contaminating protein.

As used herein, the term “nucleic acid” refers to a polymeric form of nucleotides of any length, either ribonucleotides or deoxyribonucleotides, or analogs thereof. This term refers to the primary structure of the molecule, and thus includes double-and single-stranded DNA, as well as double- and single-stranded RNA. It also includes modified nucleic acids such as methylated and/or capped nucleic acids, nucleic acids containing modified bases, backbone modifications, and the like.

As used herein, “SWISS-MODEL” refers to a fully automated protein structure homology-modelling server for homology modeling of 3D protein structures, accessible via the Expasy web server, or from the program DeepView (Swiss Pdb-Viewer). The SWISS-MODEL consists of three integrated components: (1) The SWISS-MODEL pipeline—a suite of software tools and databases for automated protein structure modeling; (2) The SWISS-MODEL Workspace—a web-based graphical user workbench; and (3) The SWISS-MODEL Repository—a continuously updated database of homology models for a set of model organism proteomes of high biomedical interest. Using the SWISS-MODEL pipeline comprises four main steps that are involved in building a homology model of a given protein structure: (1) Identification of structure template(s). BLAST and HHblits are used to identify templates. The templates are stored in the SWISS-MODEL Template Library (SMTL), which is derived from PDB. (2) Alignment of target sequence and template sequence(s). (3) Model building and energy minimization. SWISS-MODEL implements a rigid fragment assembly approach for modeling. (4) Assessment of the model's quality using QMEAN, a statistical potential of mean force.

The present disclosure provides enzymes and biocatalytic processes for generating dihydrofurans and downstream products such as 5-hydroxymethyl-2-furoic acid (HMFA), 2,5-furandicarboxylic acid (FDCA), furan dicarboxylic methyl ester (FDME), and polyethylene 2,5-furandicarboxylate (PEF). FDME is a methyl ester of FDCA and a derivative that can be polymerized with ethylene glycol to produce PEF. Also provided are methods comprising biocatalytic processes for generating a dihydrofuran by contacting a substrate keto-sugar with a glycoside hydrolase, thereby producing a dihydrofuran. The dihydrofuran can be further processed to produce HMFA. In some embodiments, the HMFA can also be further processed. chemically or biocatalytically, to generate FDCA. FDCA can be utilized to generate PEF.

Also provided are isolated and/or modified glycoside hydrolases that can be used to effectuate the dehydration of keto-sugars. such as 2-keto-3-deoxy gluconate (KDG).

### Glycoside Hydrolases

Provided herein are one or more glycoside hydrolases or motifs thereof. Glycoside hydrolases are enzymes that can hydrolyze a glycosidic bond between carbohydrates or between a carbohydrate and a non-carbohydrate moiety. In some embodiments, glycoside hydrolases can be utilized in a biocatalytic reaction to dehydrate a substrate, such as KDG.

Glycosyl hydrolases are grouped into families, based on sequence similarity. This classification is available on the CAZy (CArbohydrate-Active EnZymes) web site. Because the fold of proteins is better conserved than their sequences, some of the families can be grouped in ‘clans’. In some embodiments, a glycoside hydrolase can be a part of any of the 128 families of glycosyl hydrolases and/or a part of any of the identified clans.

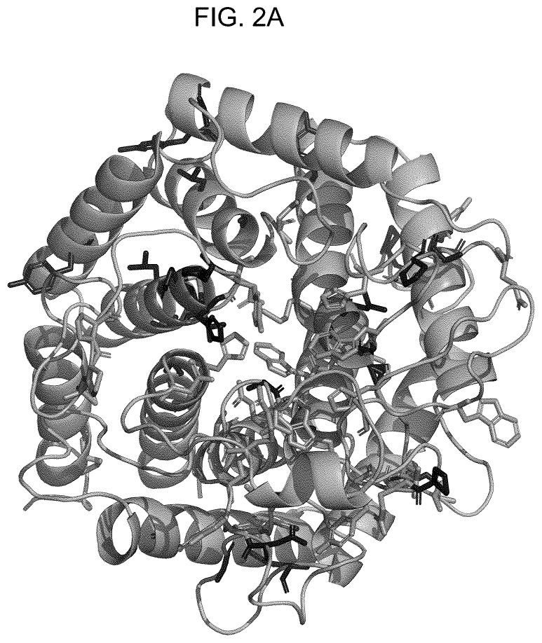

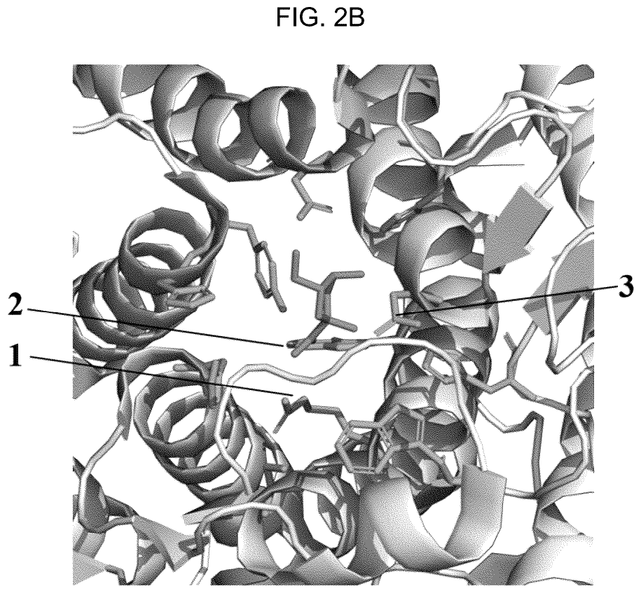

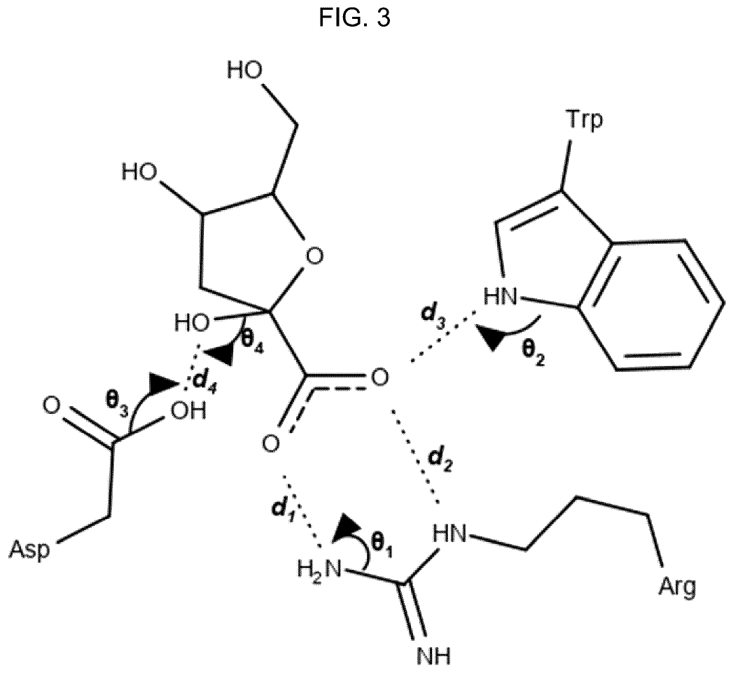

In some embodiments, a glycoside hydrolase is from the GH88 and/or GH105 family. In some embodiments, the glycoside hydrolase has the classification of E.C3.2.1.179 and/or E.C3.2.1.172. In some embodiments, a glycoside hydrolase comprises a geometry and/or active site as provided in FIG. 2A, FIG. 2B, and/or FIG. 3.

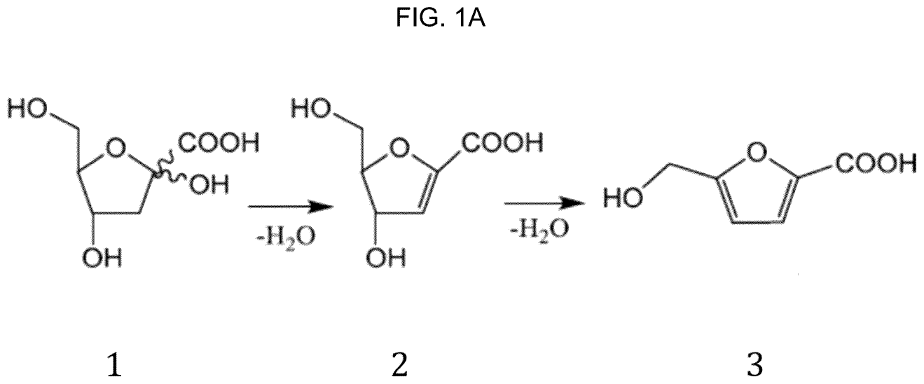

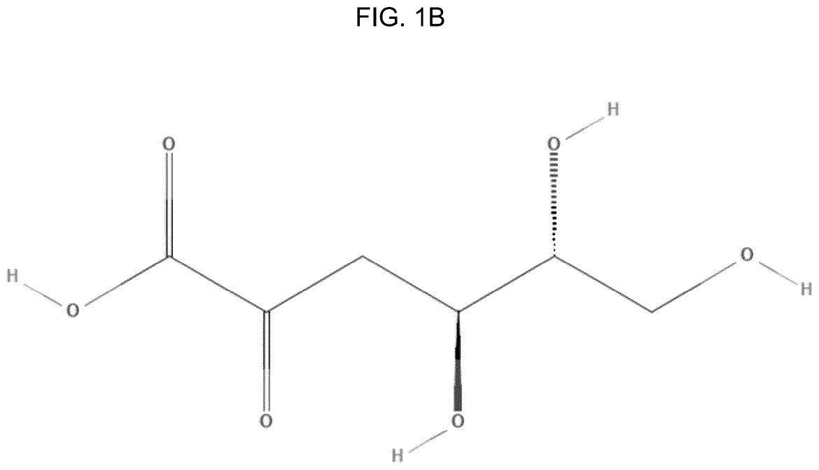

In some embodiments, computational methods can be utilized to identify and/or design geometries of glycoside hydrolases capable of effectuating a dehydration reaction with a substrate, such as KDG. A linear representation of KDG is shown in FIG. 1B. An example of such a reaction is shown in FIG. 1A. The biological dehydration reaction of keto-sugars (eg, KDG 1) to a dihydrofuran (e.g. FDCA 2) can be performed via glycosyl hydrolase (with further dehydration to HMFA 3). Protonation of the hydroxyl group of KDG 1, in order to sufficiently activate it for leaving, occurs by aspartic acid, in an example. With reference to FIG. 2A and FIG. 2B, the overall structural fold of the glycosyl hydrolase enzymes is an (alpha/alpha) 6 fold are partially shown. Moreover, FIG. 2B illustrates residues of the glycosyl hydrolase that are key to the reaction. For instance, aspartic acid 3 can act as a general acid/base. In a first step, the aspartic acid 3 acts as an acid to provide a proton to the leaving hydroxyl group at the anomeric carbon C1 of the substrate (e.g., KDG). The aspartic acid 3 (i.e., the catalytic residue), is situated about 3.5 Å from the anomeric carbon C1 of the substrate. During a second step, the aspartic acid 3 acts as a base to extract a proton from anomeric carbon C2. thus facilitating the dehydration reaction. The substrate binding occurs via interactions of the keto-sugar carboxylate group on C1 with arginine 1 and tryptophan 2 residues in the active site.

In some embodiments, a glycoside hydrolase comprises an active site geometry set forth in Table 1. Table 1, with reference to FIG. 3, provides an exemplary active site/residue geometry for a glycoside hydrolase polypeptide useful for KDG dehydration. For instance, Table 1 describes distances (e.g., d₁, d₂, d₃, d₄) and angles (e.g., θ₁, θ₂, θ₃, θ₄) at each of the key residues and as illustrated in FIG. 3.

In some embodiments, Residue #1 and Residue #2 of Table 1 coordinate the carboxylate group at anomeric carbon C₁ of KDG within the active site. In some embodiments, Residue #1 and Residue #2 are arginine (Arg) and tryptophan (Trp), respectively. In some embodiments, Residue #3 is catalytic and serves as a proton donor which adds the proton to the leaving hydroxyl group at anomeric carbon C1 of KDG. As indicated above, catalytic Residue #3 is about 3.5 Å away from the anomeric carbon C1 with leaving hydroxyl group. In some embodiments, Residue #1, Residue #2, and Residue #3 perform glycoside hydrolase activity in a KDG dehydration reaction.

TABLE 1
Exemplary geometry of glycoside hydrolases for KDG dehydration

| | RESIDUE #1 | RESIDUE #2 | RESIDUE #3 |
|---|---|---|---|
| | Arg | Trp | Asp/Glu |
| Distance | COO—NH2 (d₁) = | COO—NE1 (d₃) = | OH—OD2 (d₄) = |
| (in Angstroms) | 2.7 ± 1.5 | 3.0 ± 1.5 | 3.0 ± 0.2 |
| | COO—NE (d₂) = | | |
| | 3.0 ± 1.5 | | |
| Angle | CZ—NH2—OOC (θ₁) = | CE2—NE1—OOC (θ₂) = | CG—OD2—OH (θ₃) = |
| (in Degrees) | 120 ± 30 | 115 ± 30 | 120 ± 30 |
| | | | C1—OH—OD2 (θ₄) = |
| | | | 109 ± 30 |
| Dihedral | | | CG—OD2—OH—C1 = |
| (in Degrees) | | | 180 ± 30 |

In some embodiments, glycoside hydrolases that comprise a substantially similar geometry as provided in Table 1 is also contemplated. For example, a glycoside hydrolase can also comprise residues, such as Residue #1, Residue #2, and Residue #3, that are within about 1.5, 1, 0.9, 0.8, 0.7, 0.6, 0.5, 0.4, 0.3, 0.2, or 0.1 Angstroms of their values shown in Table 1. In some embodiments, a glycoside hydrolase comprises residues, such as Residue #1, Residue #2, and Residue #3, within at least 0.5 Angstroms of their value shown in Table 1. Similarly, for example, a glycoside hydrolase may comprise, residues, such as Residue #1, Residue #2, and Residue #3, that are within about 1.0, 2.5, 5.0, 10, 15, 30, 45, 60, 90, 120, 150, and 180 degrees of their values shown in Table 1.

In embodiments, provided are glycoside hydrolases that comprise the geometry set forth in Table 1. In embodiments, provided are also isolated polypeptides that code for glycoside hydrolases that comprise the geometry set forth in Table 1.

### Motifs of Glycoside Hydrolases

Also provided are motifs of the described glycoside hydrolases. By “motif” it is intended to refer to a portion of the polynucleotide or a portion of the amino acid sequence. Motifs may retain activity toward KDG dehydration. Thus, motifs of a polynucleotide sequence may range from at least about 2 nucleotides, about 10 nucleotides, about 20 nucleotides, about 50 nucleotides, about 100 nucleotides, and up to the full-length polynucleotide sequence corresponding to the glycoside hydrolase. In some embodiments, a motif of a glycoside hydrolase is at least about 2, 5, 8, 10, 35, 60, 85, 110, 135, 160, 185, 210, 235, 260, 285, 310, 335, 360, 385, 410, 435, 460, 485, 510, 535, 560, 585, 610, 635, 660, 685, 710, 735, 760, 785, 810, 835, 860, 885, 910, 935, 960, 985, or up to about 1000 amino acid residues, or up to about the total number of amino acid residues present in a full-length glycoside hydrolase, such as any of SEQ ID NO: 1-116.

In some embodiments, a motif of a glycoside hydrolase comprises a biologically active portion of the glycoside hydrolase capable of at least partially dehydrating KDG. In some embodiments, a motif comprises a residue geometry as set forth in any of Table 1 or having a substantially similar residue geometry.

In some embodiments, a motif of a glycoside hydrolase can be prepared by isolating a portion of one of the polynucleotides encoding a polypeptide capable of KDG dehydration, expressing the encoded portion of the polypeptides capable of KDG dehydration (e.g., by recombinant expression in vitro), and assaying for KDG dehydration activity.

In some embodiments, a glycoside hydrolase is provided in any form. In some embodiments, the glycoside hydrolase is in DNA, RNA, protein, or in combinations thereof. In some embodiments, the glycoside hydrolase is provided in an isolated form. In some embodiments, the glycoside hydrolase is provided as a whole cell system.

In some embodiments, provided herein is a recombinant glycoside hydrolase polypeptide comprising an amino acid sequence that is at least 10% to at least 99.73% identical to an amino acid sequence selected from the group consisting of SEQ ID NOs: 1-116. In some embodiments, the recombinant glycoside hydrolase polypeptide comprises an amino acid sequence selected from the group consisting of SEQ ID NOs: 1-116. In some embodiments, the glycoside hydrolase polypeptide further comprises a tag amino acid sequence. In some embodiments, the tag amino acid sequence is His6.

In some embodiments, a composition that comprises a glycoside hydrolase is at least partially pure. In some embodiments, a composition that comprises a glycoside hydrolase is substantially pure. The degree of purity of the glycoside hydrolase may vary, e.g., it may be provided as a crude, semi-purified, or purified enzyme preparation. In some embodiments, the glycoside hydrolase polypeptide is free of impurities.

### Modified Glycoside Hydrolases

The present disclosure also provides modified glycoside hydrolases. In some embodiments, a glycoside hydrolase provided herein comprises one or more modifications. Modifications can be of any region of a glycoside hydrolase. In some embodiments, a modification is within an active site. A modification to an active site can confer upon the glycoside hydrolase increased binding and/or catalytic efficiency to a substrate, such as KDG. In some embodiments, a modified glycoside hydrolase provided herein comprises a modification around a catalytic residue to recognize, bind, and/or be more catalytically efficient towards KDG. In some embodiments, glycoside hydrolases are modified to comprise the geometry set forth in Table 1 or a substantially similar geometry thereto. In some embodiments, a glycoside hydrolase is modified to improve upon the geometry set forth in Table 1 in order to increase recognition, binding, and/or catalytic efficiency towards a substrate such as KDG. Such modifications can be informed by the experimental results shown in Table 3. Varying reaction conditions were evaluated for each of SEQ ID NO: 1-116 and reaction results were assessed.

A modified glycoside hydrolase can be generated using any means. In some embodiments, a nucleotide sequence or amino acid sequence is modified to generate a recombinant glycoside hydrolase. In some embodiments, an amino acid sequence is modified. Modifications comprise one or more, substitutions, deletions, insertions, and any combination thereof. Modifications can comprise use of natural amino acid residues, synthetic amino acid residues, or combinations thereof. In some embodiments, a modification comprises a substitution.

In some embodiments, a polynucleotide encoding a glycoside hydrolase polypeptide is modified. A modified polynucleotide can comprise a deletion. In some cases, a deletion is a base truncation at the 5′ and/or 3′ end and/or a deletion of one or more nucleotides at one or more internal sites within the native polynucleotide. In some cases, a modification comprises an insertion of one or more bases at any of the 5′, 3′, and/or one or more internal sites of the polynucleotide. In some embodiments, a modification comprises a substitution of one or more nucleotides at one or more sites in a polynucleotide. In the case of polynucleotides, modifications can comprise conservative modifications. A conservative modification can comprise an an amino acid replacement in a protein that changes a given amino acid to a different amino acid with similar biochemical properties (e.g. charge, hydrophobicity, and/or size). In some embodiments, a conservative modification comprises those sequences that, because of the degeneracy of the genetic code, encode an amino acid sequence of any of the polypeptides capable of carrying out KDG dehydration.

In some embodiments, a modified glycoside hydrolase refers to a modified sequence encoding a polypeptide or protein. Protein modifications can comprise deletions, truncations, additions, substitutions, or combinations thereof. In some embodiments, a glycoside hydrolase protein is modified by truncation at either of the 5′ and/or 3′ end. In some embodiments, a glycoside hydrolase encoding or coding sequence is modified by the addition, deletion, or both of one or more residues at any of the 5′, 3′, and/or internal region. A modified glycoside hydrolase can retain biological activity. In some embodiments, a modified glycoside hydrolase retains comparable biological activity as compared to an unmodified glycoside hydrolase. In some cases, the biological activity can be reduced. In some cases, the biological activity can be increased by way of the modification.

In some embodiments, a modified glycoside hydrolase comprises a sequence having at least 80%, 85%, 90%, 95%, 96%, 97%, 98%, 99%, or 100% identity to a sequence selected from SEQ ID NO: 1-116, active variants thereof, fragments thereof, modified versions thereof. In some embodiments, a modified glycoside hydrolase comprises an amino acid sequence that is at least 10% to at least 99.73% identical to an amino acid sequence selected from the group consisting of SEQ ID NOs: 1-116. In some embodiments, a modified glycoside hydrolase comprises an amino acid sequence that is at least 82% identical to SEQ ID NO: 1. In some embodiments, a modified glycoside hydrolase comprises an amino acid sequence that is at least 88% identical to SEQ ID NO: 19. In some embodiments, a modified glycoside hydrolase comprises an amino acid sequence that is at least 85%, 87%, 89%, 91%, 93%, 95%, 97%, 99%, or 100% identical to SEQ ID NO: 27. In some embodiments, computationally designed glycoside hydrolases comprise SEQ ID NOs: 35-116.

In some embodiments, a polynucleotide that encodes for a modified glycoside hydrolase is also provided. In some embodiments, provided is a polynucleotide that encodes for a modified glycoside hydrolase that comprises any one of SEQ ID NO: 1-116.

In some embodiments, a glycoside hydrolase or a motif thereof comprises KDG dehydration activity and comprises an active site having a catalytic residue geometry as set forth in Table 1 or having a substantially similar catalytic residue geometry. In some embodiments, the glycoside hydrolase or motif thereof that comprises the KDG dehydration activity and comprises the active site having catalytic residues geometry as set forth in Table 1 further comprises an amino acid sequence having at least 10%, 20%, 30%, 40%, 75% 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%. 97%, 98%, 99%, or 100% percent identity to any one of SEQ ID NOs: 1-116.

In some embodiments, a glycoside hydrolase comprises an active site having a catalytic

residue geometry as set forth in any of Table 1, or having a substantially similar catalytic residue geometry and further comprises: (a) an amino acid sequence having at least 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 95%, 96%, 97%, 98%, 99%, or 100% sequence identity to any one of SEQ ID NOS: 1-34, wherein (i) the amino acid residue in the encoded polypeptide that corresponds to amino acid position 40 of SEQ ID NO: 1 comprises histidine or glycine or cystine or serine or tyrosine or phenylalanine or isoleucine or asparagine or glutamine or aspartic acid or glutamic acid; (ii) the amino acid residue in the encoded polypeptide that corresponds to amino acid position 41 of SEQ ID NO: 1 comprises tyrosine or tryptophan; (iii) the amino acid residue in the encoded polypeptide that corresponds to amino acid position 42 of SEQ ID NO: 1 comprises histidine or threonine or methionine or proline or glycine or glutamic acid or glutamine or asparagine or isoleucine or leucine or valine or serine or tryptophan; (iv) the amino acid residue in the encoded polypeptide that corresponds to amino acid position 88 of SEQ ID NO: 1 comprises aspartic acid or leucine or isoleucine or phenylalanine or asparagine or lysine; (v) the amino acid residue in the encoded polypeptide that corresponds to amino acid position 132 of SEQ ID NO: 1 comprises histidine or alanine or leucine or valine or serine or cystine or proline or aspartic acid or glutamic acid or asparagine or arginine or glycine or glutamine; (vi) the amino acid residue in the encoded polypeptide that corresponds to amino acid position 146 of SEQ ID NO: 1 comprises tyrosine or phenylalanine or methionine; (vii) the amino acid residue in the encoded polypeptide that corresponds to amino acid position 147 of SEQ ID NO: 1 comprises methionine or alanine or leucine or phenylalanine; (viii) the amino acid residue in the encoded polypeptide that corresponds to amino acid position 189 of SEQ ID NO: 1 comprises histidine or arginine or glutamine or valine or alanine; (ix) the amino acid residue in the encoded polypeptide that corresponds to amino acid position 211 of SEQ ID NO: 1 comprises tryptophan; (x) the amino acid residue in the encoded poly peptide that corresponds to amino acid position 214 of SEQ ID NO: 1 comprises serine or alanine or glycine; (xi) the amino acid residue in the encoded polypeptide that corresponds to amino acid position 220 of SEQ ID NO: 1 comprises methionine or tyrosine or valine or leucine or alanine or glycine or phenylalanine; (xii) the amino acid residue in the encoded polypeptide that corresponds to amino acid position 278 of SEQ ID NO: 1 comprises serine; (xiii) the amino acid residue in the encoded polypeptide that corresponds to amino acid position 282 of SEQ ID NO: 1 comprises leucine or methionine or isoleucine or valine or phenylalanine or threonine or glycine or glutamine; (xiv) the amino acid residue in the encoded polypeptide that corresponds to amino acid position 352 of SEQ ID NO: 1 comprises histidine or tyrosine or tryptophan or phenylalanine or lysine or valine or arginine; (xv) the amino acid residues in the encoded polypeptide that corresponds to amino acid positions 211-217 of SEQ ID NO: 1 comprises a fragment of W(A/G/S/T) R (G/A/S) (N/Q/I/L/M)(G/A/T)W; (xvi) the amino acid residues in the encoded polypeptide that corresponds to amino acid positions 141-145 of SEQ ID NO: 1 comprises a fragment of (W/I) (L/I/C/A/V/S) D (G/D/A/C/T/V/I/N) (L/M/I/V); and/or, (xvii) the amino acid residue in the encoded protein that corresponds to the amino acid position of SEQ ID NO: 1 as set forth in Table 1 and corresponds to the specific amino acid substitution also set forth above in (d)(i)-(d) (xvii) or any combination of these residues.

In some embodiments, a glycoside hydrolase comprises a point mutation in SEQ ID NO: 1 selected from the group consisting of: H42I, H42T, H42V, H42W, D88C, D88N, H132E, K133M, W141Y, H189A, H189V, V331K, G332E, G332M, G332V, S334A, S334C, S334D, S334E, S334G, S334I, S334K, S334M, S334N, S334Q, S334R, S334T, S334V, A335V, A335P, A335L, A335C, H352R, and H352V. In some embodiments, a glycoside hydrolase comprises a modification of a residue selected from the group consisting of 332, 334, and 335 of SEQ ID NO: 1. In some embodiments, a glycoside hydrolase comprises a modification in the loop region. In some embodiments, a modification of a loop region comprises any one of the residues at amino acid position nos. 331-336 of SEQ ID NO: 1. In some embodiments, a glycoside hydrolase comprises a point mutation in SEQ ID NO: 19 selected from the group consisting of: D41A, D41C, D41E, D41N, D41S, D41T, H87A, H87C, H87E, H87G, H87Q, H87R, H87S, L152M, L152N, W225Y, S337A, Y338V, H339A, H339N, W352F, Y356A, Y356C, Y356F, and Y356H. In embodiments, a point mutation confers increased activity as compared to an otherwise comparable glycoside hydrolase that lacks the point mutation.

In some embodiments, a glycoside hydrolase provided herein may not comprise catalytic residue geometry as set forth in any of Table 1 but retains KDG dehydration activity.

In some embodiments, provided is an isolated polypeptide that comprises a sequence that codes for any of the provided glycoside hydrolases. In embodiments, the isolated polypeptide comprises: a first motif that binds 2-keto-3-deoxygluconate and a second motif that comprises a catalytic residue. In embodiments, the first motif comprises at least two residues, wherein the first residue comprises arginine, wherein the second residue comprises tryptophan, phenylalanine, or tyrosine. In embodiments, the second residue comprises tryptophan In embodiments, the second residue comprises phenylalanine. In embodiments, the second residue comprises tyrosine. In some embodiments, the catalytic residue comprises aspartic acid. In some embodiments, the isolated polypeptide is a homolog to SEQ ID NO: 1 or SEQ ID NO: 19 as determined by SWISS-MODEL homology modeling. In some embodiments, the isolated polypeptide comprises at least about 25%, 35%, 45%, 55%, 65%, 75%, 85%, 95%, 97%, or 100% identity to SEQ ID NO: 1 or SEQ ID NO: 19.

In embodiments, provided is an isolated polypeptide that comprises a sequence that codes for any of the provided glycoside hydrolases. In embodiments, the isolated polypeptide comprises a first motif that binds 2-keto-3-deoxygluconate and a second motif that comprises a catalytic residue (or vice versa). In embodiments, the first motif comprises at least two residues. The at least two residues may be arginine and one of tryptophan, phenylalanine, and tyrosine. In embodiments, the second residue of the first motif may be tryptophan. In embodiments, the second residue of the first motif may be phenylalanine. In embodiments, the second residue of the first motif may be tyrosine. In some embodiments, the catalytic residue of the second motif comprises aspartic acid or glutamic acid. In embodiments, the catalytic residue of the second motif is aspartic acid. In some embodiments, the isolated polypeptide is a homolog to SEQ ID NO: 1 or SEQ ID NO: 19 as determined by SWISS-MODEL homology modeling. In some embodiments, the isolated polypeptide comprises at least about 25%, 35%, 45%, 55%, 65%, 75%, 85%, 95%, 97%, or 100% identity to SEQ ID NO: 1 or SEQ ID NO: 19.

In embodiments, alanine scanning, interchangeable with other site-directed mutagenesis techniques, can be used to determine the contribution of specific residues to stability and function of a modified glycoside hydrolase. Results of these analyses are reflected throughout this document.

In embodiments, a glycoside hydrolase does not comprise a modification to SEQ ID NO: 1 selected from the group consisting of: D88 (G/I/L/N/Q/A/R/V/W/Y), H132 (M/P/R/T/W/K/Y/D/C/G), H189 (F/I/L/N/Y/T/V/S), H352 (W/G/E/K/Q), H40 (I/K/L/P/R/W), H42 (C/K/Q/S), M147 (A/D/F/K/N/H/S/V/E/G/L/P/I/Q/T/Y/C/W), W141 (A/D/H/K/M/P/R/T/Y/E/I/N/Q/S/V/C/F), W211 (E/L/V/A/F/M/R/G/I/K/N/Q), and Y41 (A/D/F/M/R/G/C/E/H/K/N/P/Q/V/I/T).

According to an embodiment, the isolated polypeptide comprises a first motif that binds KDG and a second motif comprising a catalytic residue. In an embodiment, the first motif and the second motif are separated by about 70 residues. In an embodiment, an arginine of the first motif and a catalytic residue of the second motif are separated by 70 residues. In an embodiment, an arginine of the first motif and a catalytic residue of the second motif are separated by 72 residues.

In an embodiment, the first motif may be at least 5 residues, at least 10 residues, at least 15 residues, at least 20 residues, at least 30 residues, at least 50 residues, at least 100 residues, and the like. In an embodiment, the first motif may be 5 residues and have a sequence of RxQTW, where R is arginine, x is a serine or an aliphatic amino acid such as glycine, alanine, valine, leucine, isoleucine, and proline, Q is glutamine. Tis threonine, W is tryptophan, and R and W are the substrate binding residues, independently and in combination. In an embodiment, the first motif may be 10 residues and have a sequence of Rx₁QTW(2x₂)Yx₂Y, where R is arginine, x₁ is serine, x₂ is an aliphatic amino acid such as glycine, alanine, valine, leucine, isoleucine, and proline, Q is glutamine, T is threonine, W is tryptophan, Y is tyrosine, and R and W are the substrate binding residues, independently and in combination.

In an embodiment, the first motif may be at least 5 residues, at least 10 residues, at least 15 residues, at least 20 residues, at least 30 residues, at least 50 residues, at least 100 residues, and the like. In an embodiment, the second motif may be 2 residues and have a sequence of xD, where x is an aliphatic amino acid such as glycine, alanine, valine, leucine, isoleucine, and proline, and D is asparatic acid, D being a catalytic residue. In an embodiment, the second motif may be 18 residues and have a sequence of (2x)KSE(3x)DT(2M)xSxPFx, where x is an aliphatic amino acid such as glycine, alanine, valine, leucine, isoleucine, and proline, K is lysine, S is serine, E is glutamic acid, D is asparatic acid, T is threonine, M is methionine, P is proline, and F is phenylalanine, D being a catalytic residue.

In an embodiment, the modified glycoside hydrolase comprises a sequence of, as formula 1, P(19x)LPP(4x)HYHQGVxLxG(4x)(W/Y)(10x)Y(3x)(Y/W)x(D/E)(6x)G(9x)D(2x)Q(P/A)Gx (L/I)L(2x)L(7x)(R/K)Y(2x)(A/G)(3x)(L/I)(9x)(T/N)xE(G/Q)G(F/Y)(W/F)H(K/N)(3x)Px(Q/E) (M/Q)WLDGLYMxG(5x)Y(A/G)(9x)D(4x)Q(6x)(H/K)(T/M)(R/K)(3x)TGL(2x)H(A/G)(W/F) (D/S)(2x)(R/K)(3x)W(A/S)(D/N)(2x)(T/S)Gx(S/A)PExW(G/A)R(S/A)xGW(9x)(D/E)x(I/L) P(2x)H(20x)Q(4x)GxWxQ(V/I)x(D/N)(K/R)(G/V)(4x)NW(L/P)ExSx(S/T)xL(6x)K(G/A) (15x)(K/Q)(A/G)(F/Y)xG(18x)(I/V)C(I/V)GT(S/G)xGxY(5x)R(5x)D(L/M)HG(V/A)GA(F/L), wherein formula 1 has at least 50% homology to SEQ ID NO: 1 and wherein x is any amino acid. In an embodiment, the modified glycoside hydrolase has at least 55%, 60%, 70%, 80%, 90%, 95%, 97%, 98%, 99%, or 100% homology to SEQ ID NO: 1. In an embodiment, the modified glycoside hydrolase comprises Y at residue 41, D at residue 8, H at residue 132, W at residue 141, D at residue 143, M at residue 147, H at residue 189, W at residue 211, G at residue 212, R at residue 213, W at residue 217, S at residue 278, L or M at residue 282, C at residue 330, and H or K at residue 352, wherein the residues are numbered according to SEQ ID NO: 1. In an embodiment, the modified glycoside hydrolase comprises a loop region having at least 21 residues. In an embodiment, the modified glycoside hydrolase comprises a loop region having at least one modification selected from the group consisting of 331 through 336 of SEQ ID NO: 1. In an embodiment, the modified glycoside hydrolase comprises the at least one modification selected from the group consisting of 331 through 336 of SEQ ID NO: 1, including one or more of V331K, G332E, G332M, G332V, S334A, S334C, S334D, S334E, S334G, S334I, S334K, S334M, S334N, S334Q, S334R, S334T, S334V, A335V, A335P, A335L, and A335C.

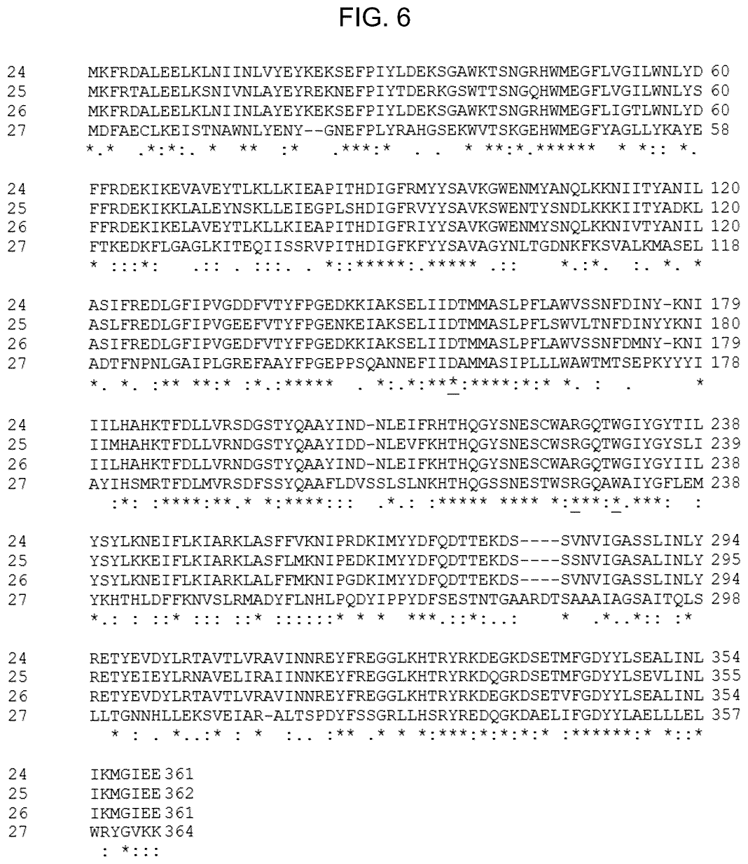

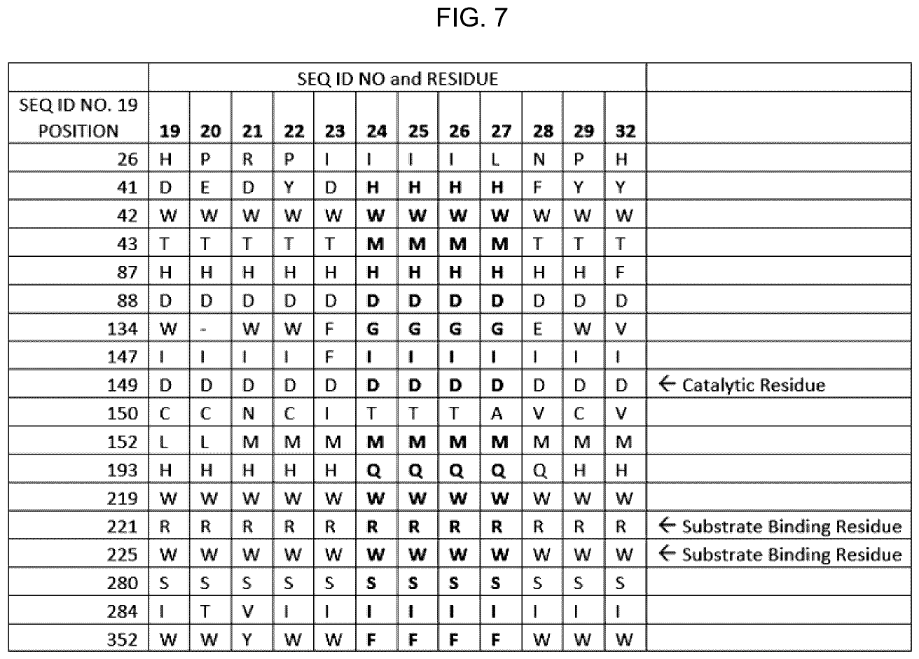

In an embodiment, the modified glycoside hydrolase comprises a sequence of, as formula 2, (F/Y)P(8x)(W/Y)(7x)W(T/M)(2x)F(2x)G(2x)(W/Y)(2x)Y(11x)(A/G)(10x)(L/1)(8x)(H/F)D(L/T) GF(4x)(S/T)(4x)(W/Y)(15x)(A/G)(13x)(L/I)(16x)IDx(L/M)(L/M)(N/S)(22x)H(3x)(T/S)(5x) RxDxS(S/T)(6x)(D/N)(10x)TxQG(4x)SxW(A/S/T)RG(Q/L)(A/T)W(2x)YG(28x)PxD(4x)(Y/W) D(F/L)(12x)S(6x)(S/C)(33x)Y(30x)(W/F/Y)(G/A)DY(Y/F)(2x)ExL, wherein formula 2 has at least 50% homology to SEQ ID NO: 19 and wherein x is any amino acid. In an embodiment, the modified glycoside hydrolase has at least 55%, 60%, 70%, 80%, 90%, 95%, 97%, 98%, 99%, or 100% homology to SEQ ID NO: 19. In an embodiment, the modified glycoside hydrolase comprises I or L at residue 26, H at residue 41, W at residue 42, M at residue 43, H at residue 87. D at residue 88. G at residue 134, D at residue 149, T at residue 150, M at residue 152, Q at residue 193, W at residue 219, R at residue 221, W at residue 225, S at residue 280, I at residue 284, and F at residue 352, wherein the residues are numbered according to SEQ ID NO:19. In an embodiment, the modified glycoside hydrolase comprises a sequence selected from, or having homology to, the group consisting of SEQ ID NOS: 24 to 27. An alignment of SEQ ID NOS: 24 to 27 is shown in FIG. 6, wherein residues denoted * correspond to a catalytic residue and substrate binding residues, as outlined above. The same is presented in FIG. 7, with further context to other sequences possessing the same residues at similar positions relative to SEQ ID NO: 19.

In an embodiment. the modified glycoside hydrolase comprises a sequence of, as formula 3, (W/Y)7x(A/G)(92x)WxD(35x)(L/M)(9x)(H/R)(22x)W(A/G/S)R(2x)(G/S)W(8x)(L/1)(27x)Q(3x)(G/K)xW(3x)(I/L)(9x)ExSx(S/T)(9x)(A/G)(52x)Gx(G/A), wherein formula 3 has at least 50% homology to SEQ ID NO: 2. In an embodiment, the modified glycoside hydrolase has at least 55%, 60%, 70%, 80%, 90%. 95%, 97%, 98%, 99%, or 100% homology to SEQ ID NO: 2.

### Modification Methodology

In some embodiments, glycoside hydrolase polypeptides and/or a motif thereof may be modified using one or more methodologies. In some embodiments, a modification is identified through rational design modeling. Methods for such manipulations are generally known in the art. For example, amino acid sequence variants and fragments of the KDG dehydrating polypeptides can be prepared by mutations in a polynucleotide sequence. Methods for mutagenesis and polynucleotide alterations are well known in the art. See, for example, Kunkel (1985) roc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference in their entirety. Conservative substitutions, such as exchanging one amino acid with another having similar properties, are also contemplated. In some embodiments, modifications, such as mutations, that may not place the sequence out of reading frame. In some embodiments, a modification will not create complementary regions that could produce secondary mRNA structure, see, EP Patent Application Publication No. 75,444.

In some embodiments, a suitable modification can be identified with the use of well-known molecular biology techniques, as, for example, with polymerase chain reaction (PCR) and hybridization techniques, and sequencing techniques. Variant polynucleotides also include synthetically derived polynucleotides, such as those generated, for example, by using site-directed mutagenesis or gene synthesis but which still encode a polypeptide capable of KDG dehydration or through computation modeling.

In embodiments, homology modeling may be performed on designed and/or generated sequences using a SWISS-MODEL homology modeling. In embodiments, a target glycoside hydrolase sequence is utilized as a template to perform the homology modeling. In embodiments, homology modeling comprises: (a) performing a template search for related homologs; (b) ranking templates identified from (a) according to Global Model Quality Estimate (GMQE) and/or Quaternary Structure Quality Estimate (QSQE); (c) determining if top-ranked templates cover different regions of a target protein (e.g. glycoside hydrolase) and/or determining if top-ranked templates represent different conformational states: and (d) selecting a template. In embodiments, top-ranked templates can comprise templates from 1-10, 1-30, 1-50, or 1-100. In embodiments, a selected template can comprise from about 1-5%, 1-10%, 1-20%, 1-30%, 10-30%, 20-40%, 20-60%, 25-65%, 30-70%, 50-80%, 60-95%, or 25-85% identity to a target glycoside hydrolase sequence. In embodiments, a selected template can comprise at least about 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91. 93, 95. 97, 99, or 100% identity to a target glycoside hydrolase sequence. In embodiments, a selected template can comprise at least about 27% identity to a target glycoside hydrolase.

### Method of Making a Dihydrofuran

Provided is also a biocatalytic process for generating a dihydrofuran from a composition comprising a substrate keto-sugar. As used herein. “biocatalysis” or “biocatalytic” refers to the use of natural catalysts, such as protein enzymes, to perform chemical transformations on organic compounds. Biocatalysis is alternatively known as biotransformation or biosynthesis. Biocatalyst protein enzymes can be naturally occurring or recombinant proteins. In some embodiments, provided herein are also methods of making a dihydrofuran by way of dehydrating a keto-sugar. In some embodiments, a glycoside hydrolase polypeptide is capable of transforming a keto-sugar moiety to a dihydrofuran. The provided methods can comprise any of the described glycoside hydrolases, modified glycoside hydrolases, and portions thereof including but not limited to glycoside hydrolases that comprise a sequence selected from SEQ ID NO: 1-116.

The present methods can be biocatalytic, i.e., utilizes a biological catalyst. In some embodiments. the biocatalyst is protein enzyme. In some embodiments, the biocatalyst is a glycoside hydrolase polypeptide. In some embodiments, a substrate is a keto-sugar that is dehydrated by a glycoside hydrolase thereby generating a dihydrofuran.

The glycoside hydrolase, can be provided free or in an immobilized form. In some embodiments, the glycoside hydrolase preparation may be crude, semi-purified, and/or purified. In some embodiments, the glycoside hydrolase is provided as a whole-cell system, e.g., a living or non-living microbial cell, or whole microbial cells, cell lysate and/or any other form of known in the art.

In some embodiments, provided herein is a method for producing a dihydrofuran composition, comprising the steps of: (a) providing a substrate keto-sugar, such as KDG (b) contacting the keto-sugar with a glycoside hydrolase polypeptide; (c) producing a composition comprising a dihydrofuran; and (d) dehydrating the dihydrofuran at high temperature.

In some embodiments, provided herein is a method for producing a dihydrofuran

composition, comprising the steps of: (a) providing a composition comprising greater than about 0.5%, about 1%, about 2%, about 3%, about 4%, about 5%, about 10%, about 20%, about 30%, about 40%, about 50%, about 60%, about 70%, about 80%, about 90%, about 92%, about 93%, about 94%, about 95%, about 96%, about 97%, about 98%, about 99%, or about 99.6% of a substrate keto-sugar by weight on an anhydrous basis; (b) contacting the composition with a glycoside hydrolase polypeptide; (c) producing a composition comprising a dihydrofuran; and (d) dehydrating the dihydrofuran at high temperature.

### Substrate

In some embodiments, a method provided herein comprises contacting a substrate, such as a keto-sugar, with a glycoside hydrolase or motif thereof.

In some embodiments, method provided herein comprises contacting a substrate with a glycoside hydrolase or motif thereof. A substrate can comprise a keto-sugar. In some embodiments, a substrate is keto-sugar 2-keto-3-deoxy-gluconate of which the 2-keto-3-deoxy-gluconate serves as the substrate for the biotransformation with a glycoside hydrolase. A keto-sugar may be synthetic, purified (partially or entirely), commercially available, or prepared. One example of a composition useful in the method of the disclosure is chemically synthesized 2-keto-3-deoxy-gluconate brought into solution with a solvent Another example of a substrate is an enzymatically synthesized 2-keto-3-deoxy-gluconate in water. Another example of a substrate is fermented 2-keto-3-deoxy-gluconate within a broth.

In some embodiments, a composition comprises a purified substrate keto-sugar. For example, the composition may comprise greater than about 50%, about 60%, about 70%, about 80%, about 85%, about 90%, about 91%, about 92%, about 93%, about 94%, about 95%, about 96%, about 97%, about 98%, about 99%, or about 99.6% of substrate keto-sugar by weight on an anhydrous basis.

In some embodiments, a composition comprises a partially purified substrate keto-sugar. For example, the composition contains greater than about 0.5%, about 1%, about 2%, about 3%, about 4%, about 5%, about 10%, about 20%, about 30%, about 40%, or about 50%, of substrate keto-sugar by weight on an anhydrous basis.

In some embodiments, a composition comprises a substrate keto-sugar (e.g., KDG). For example, the composition may comprise between about 250 μM and about 2 M, between about 500 μM and about 1.5 M, between about 1 mM and about 1 M, between about 25 mM about 750 mM, between about 250 mM and about 500 mM, and between about 300 mM and about 400 mM of substrate keto-sugar. In an embodiment, the composition may comprise about 180 mM of substrate keto-sugar. In an embodiment, the composition may comprise about 750 mM of substrate keto-sugar. In an embodiment, the composition may comprise about 1.7 M of substrate keto-sugar.

In some embodiments, a composition comprises purified KDG. In some embodiments, the composition contains greater than about 99% KDG by weight on an anhydrous basis. In some embodiments, the composition comprises partially purified KDG. In some embodiments, the composition contains greater than about 50%, about 60%, about 70%, about 80% or about 90% KDG by weight on an anhydrous basis.

In some embodiments, provided herein is a method for producing a dihydrofuran composition, comprising the steps of: (a) providing a composition comprising a substrate keto-sugar such as KDG; (b) contacting the keto-sugar with a glycoside hydrolase polypeptide; and (c) producing a dihydrofuran. In some embodiments, the composition comprises an enzymatically produced keto-sugar. In some embodiments, the glycoside hydrolase that is utilized in a method herein is expressed in a modified microorganism.

In some embodiments, provided herein is a method for producing a dihydrofuran composition, comprising the steps of: (a) providing a composition comprising greater than about 0.5%, about 1%, about 2%, about 3%, about 4%, about 5%, about 10%, about 20%, about 30%, about 40%, about 50%, about 60%, about 70%, about 80%, about 90%, about 92%, about 93%, about 94%, about 95%, about 96%, about 97%, about 98%, about 99%, or about 99.6% of a substrate keto-sugar by weight on an anhydrous basis; (b) contacting the composition that comprises a substrate with a glycoside hydrolase polypeptide; and (c) producing a dihydrofuran. In some embodiments, the composition comprises an enzymatically produced keto-sugar. In some embodiments, the glycoside hydrolase that is utilized in a method herein is expressed in a modified microorganism.

In some embodiments, a composition comprising KDG is contacted with a glycoside hydrolase, thereby catalyzing the reaction of KDG (2-keto-3-deoxy-gluconate) to produce a dihydrofuran. In some embodiments, the composition comprises partially purified KDG. In some embodiments, the composition comprises purified KDG. In some embodiments, the composition comprises at least about >95% KDG. In some embodiments, the composition comprises at least about 95%, 96%, 97%, 98%, 99%, or 100% KDG. In some embodiments, the composition comprises greater than 0.5%, about 1%, about 2%, about 3%, about 4%, about 5%, about 10%, about 15%, about 20%, about 30%, about 40%, about 50%, about 60%, about 70% about 80%, about 90%, about 91%, about 92%, about 93%, about 94%, about 95%, about 96%, about 97%, about 98%, about 99%, or about 99.6% KDG.

The present invention also provides a method for producing a dihydrofuran composition, comprising the steps of: (a) providing a composition comprising greater than about 0.5%, about 1%, about 2%, about 3%, about 4%, about 5%, about 10%, about 20%, about 30%, about 40%, about 50%, about 60%, about 70%, about 80%, about 90%, about 92%, about 93%, about 94%, about 95%, about 96%, about 97%, about 98%, about 99%, or about 99.6% of a substrate 2-keto-3-deoxy gluconate (KDG) by weight on an anhydrous basis; (b) contacting the composition with a glycoside hydrolase; and (c) producing a composition comprising a dihydrofuran. In some embodiments, the composition comprises greater than about 0.5%, about 1%, about 2%, about 3%, about 4%, about 5%, about 10%, about 20%, about 30%, about 40%, about 50%, about 60%, about 70%, about 80%, about 85%, about 90%, about 91%, about 92%, about 93%, about 94%, about 95%, about 96%, about 97%, about 98%, about 99%, about 99.6% of the dihydrofuran 4,5-dihydro-4-hydroxy-5-hydroxymethyl-2-furancarboxylic acid (K4) by weight on an anhydrous basis.

In some embodiments, K4 can be further converted into 5-hydroxymethyl-2-furoic acid (HMFA) as described in WO2021016220. In some embodiments, HMFA can be further oxidized into FDCA using chemical oxidation described in WO2021016220 or using enzymatic oxidation, for example as described in Dijkman et al 2014, “Discovery and Characterization of a 5-Hydroxymethylfurfural Oxidase from Methylovorus sp. Strain MP688” herein incorporated by reference.

TABLE 2
Substrate and products from biocatalytic dehydration of a
keto-sugar and further oxidation

| | | Reference |
|---|---|---|
| Structure | IUPAC Name | Name |
| 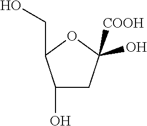 [SMILES: O=C(O)[C@]1(O)CC(O)C(CO)O1] | 2-keto-3-deoxy gluconate | KDG |
| 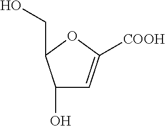 [SMILES: O=C(O)C1=CC(O)C(CO)O1] | 4,5-dihydro-4- hydroxy-5- hydroxymethyl-2- furancarboxylic acid | K4 |
| 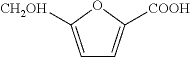 | 5-hydroxymethyl- 2-furoic acid | HMFA |
| 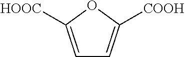 [SMILES: O=C(O)c1ccc(C(=O)O)o1] | 2,5-furan dicarboxylic acid | FDCA |
| 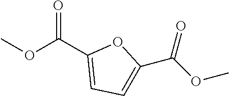 | furan dicarboxylic methyl ester | FDME |

In some embodiments, a method produces a composition comprising greater than about 80% by weight of HMFA. In some embodiments, purification produces a composition comprising greater than about 0.5%, about 1%, about 2%, about 3%, about 4%, about 5%, about 10%, about 20%, about 30%, about 40%, about 50%, about 60%, about 70%, about 80%, about 85%, about 90%, about 91%, about 92%, about 93%, about 94%, about 95%, about 96%, about 97%, about 98%, about 99%, about 99.6% by weight of HMFA. In some embodiments, the composition comprises greater than about 95% by weight of HMFA.

In some embodiments. the step of contacting the composition with the glycoside hydrolase polypeptide and the keto-sugar is at temperature between about 0.5° C. and about 75° C. In some embodiments, the temperature is from about: 0.5° C., 5° C., 10° C., 15° C., 20° C., 25° C., 30° C. 35° C., 40° C., 45° C., 50° C., 55° C. 60° C., 65° C., or 70° C., up to about 75° C. In some embodiments, the temperature is from about: −0.5° C.-5° C., 10° C.-15° C., 20° C.-30° C., 35° C.-45° C., 35° C.-50° C., 35° C.-55° C., 30° C.-60° C., or 30° C., up to about 75° C. In some embodiments, the step of contacting the composition with the glycoside hydrolase polypeptide and the keto-sugar is at temperature about 45° C.

In some embodiments, the step of contacting the composition with the glycoside hydrolase polypeptide and the keto-sugar is carried out at pH between about 3 and about 8. In some embodiments, the pH is between about 0.5 and about 1, about 1.5, about 2, about 2.5, about 3, about 3.5, about 4, about 4.5, about 5, about 5.5, about 6, about 6.5, about 7, about 7.5, and about 8. In some embodiments, the pH is between about 3 and about 6. In some embodiments, the pH is between about 3 and about 5. In some embodiments, the pH is between about 3 and about 4. In some embodiments, the pH is between about 3 and about 3.5. In some embodiments, a reduced pH results in a reduced requirement for NaOH in the contacting process. In some embodiments, the step of contacting the composition with the glycoside hydrolase and the keto-sugar is carried out at about pH 5.

In some embodiments, the step of contacting the composition with the glycoside hydrolase polypeptide and the keto-sugar is carried out in a duration of time between 1 hour and 14 days. In some embodiments, the contacting is at a duration from about 1 hour, about 3 hours, about 5 hours, about 7 hours, about 9 hours, about 11 hours, about 13 hours, about 15 hours, about 17 hours, about 19 hours, about 21 hours, about 23 hours, about 25 hours, about 27 hours, about 29 hours, about 31 hours, about 33 hours, about 35 hours, about 37 hours, about 39 hours, about 41 hours, about 43 hours, about 45 hours, about 48 hours, about 49 hours, about 51 hours, about 53 hours, about 55 hours, about 57 hours, about 59 hours, about 61 hours, about 63 hours, about 65 hours, about 67 hours, about 69 hours, about 71 hours, or about 73 hours, up to about 75 hours. In some embodiments, the step of contacting the composition with the glycoside hydrolase is carried out in a duration of time of about 48 hours. In some embodiments, the contacting is at a duration from about 1 day to about 14 days. In some embodiments, the contacting is at a duration of from about 1 day, 2 days, 3 days, 4 days, 5 days, 6 days, 7 days, 8 days, 9 days, 10 days, 11 days,. 12 days, or about 13 days, up to about 15 days. In some embodiments, the contacting is at a duration from about 4 days to about 10 days. In some embodiments, the step of contacting the composition with the glycoside hydrolase is carried out in a duration of time of about 6 days.

In some embodiments, the resulting composition that comprises K4 comprises greater than about 0.5%, about 1%, about 2%, about 3%, about 4%, about 5%, about 10%, about 20%, about 30%, about 40%, about 50%, about 60%, about 70%, about 80%, about 85%, about 90%, about 91%, about 92%, about 93%, about 94%, about 95%, about 96%, about 97%, about 98%, about 99%, about 99.6% of the K4 by weight on an anhydrous basis.

In some embodiments, at least about 5%, about 10%, about 20%, about 30%, about 40%, about 50%, about 60%, about 70%, about 80%, or about 90% of the substrate KDG in the composition is converted to K4.

The reaction medium for conversion can be aqueous. In some embodiments, a reaction medium can be purified water, buffer, or a combination thereof. In some embodiments, the reaction medium is a buffer. Suitable buffers include, but are not limited to, acetate buffer, citrate buffer, phosphate buffer, and Bis-Tris buffer. In some embodiments, the reaction medium is acetate buffer. In some embodiments, the reaction medium is phosphate buffer. In some embodiments, the reaction medium is Bis-Tris buffer. The reaction medium can also be, alternatively, an organic solvent. In some embodiments, the reaction medium is supplemented with glycerol, Tween-20, sucrose, or sorbitol as enzyme stability agents. In some embodiments, the reaction medium is supplemented with 20% glycerol or 0.1% Tween or 2M sucrose or 2M sorbitol.

In some embodiments, the conversion of KDG to a dihydrofuran is at least about 2% complete, as determined by any of the methods mentioned above. In some embodiments, the conversion of KDG to a dihydrofuran is at least about 10% complete, at least about 20% complete, at least about 30% complete, at least about 40% complete, at least about 50% complete, at least about 60% complete, at least about 70% complete, at least about 80% complete, at least about 90% complete, at least about 95% complete, at least about 100% complete. In some embodiments, the conversion of KDG to a dihydrofuran is greater than about 80% complete. In some embodiments, at least about 5%, about 10%, about 20%, about 30%, about 40%, about 50%, about 60%, about 70%, about 80%, or about 90% of the KDG in the composition is converted to dihydrofurans.

In some embodiments, a reaction can be monitored by a means including but not limited to: HPLC, LCMS, TLC, IR, UV, or NMR. In embodiments, a reaction is monitored using LCMS, UV, or both LCMS and UV.

In some embodiments, contacting of the composition with the glycoside hydrolase and/or glycoside hydrolase polypeptide and the KDG can be carried out in a duration of time between 1 hour and 14 days, such as, for example, about 1 hour, about 6 hours, about 12 hours, about 24 hours, about 48 hours, about 72 hours, about 120 hours, about 3 days, about 4 days, about 5 days, about 6 days, about 7 days, about 8 days, about 9 days, or about 10 days. In some embodiments, the reaction is carried out for about 6 days. In some embodiments, the reaction is carried out for about 7 days.

### Conversion of Dihydrofuran to HMFA and FDCA

In some embodiments, a dihydrofuran further undergoes processing, such as purification and/or dehydration, to produce HMFA and/or FDCA, for example as described in FIG. 1A, wherein HMFA is indicated at 3. In some embodiments, dihydrofuran is chemically converted to HMFA using acidic conditions. In some embodiments, the HMFA is purified. In some embodiments, the HMFA is purified before proceeding with a subsequent reaction.

In some embodiments, provided herein is a method for producing HMFA, comprising the steps of: (a) providing a composition comprising greater than about 0.5%, about 1%, about 2%, about 3%, about 4%, about 5%, about 10%, about 20%, about 30%, about 40%, about 50%, about 60%, about 70%, about 80%, about 90%, about 92%, about 93%, about 94%, about 95%, about 96%, about 97%, about 98%, about 99%, or about 99.6% of a substrate keto-sugar by weight on an anhydrous basis: (b) contacting the composition with a glycoside hydrolase polypeptide: (c) producing a composition comprising a dihydrofuran: and (d) dehydrating the dihydrofuran to HMFA under acidic conditions. In embodiments, also provided is a method comprising contacting a composition that comprises HMFA with a glycoside hydrolase polypeptide thereby generating a dihydrofuran. In embodiments, the dihydrofuran is dehydrated to HMFA under acidic conditions.

In some embodiments, dihydrofuran is converted into HMFA at high temperature. In some embodiments, the step of contacting the composition with the glycoside hydrolase polypeptide and the KDG can be carried out at temperature between about 0.5° C. and about 110° C., such as, for example, about 10° C., about 20° C., about 30° C., about 40° C., about 50° C., about 60° C., about 70° C., about 80° C., about 90° C., about 100° C., or about 110° C. In a preferred embodiment, the reaction is carried out at about 74° C. In some embodiments. the reaction is carried out at about 69° C. In another embodiment, the reaction is carried out at about 63° C.

In some embodiments, dihydrofuran contacted with an acid to effectuate its conversion to HMFA. The acid may be selected from inorganic acids, such as from hydrochloric acid, sulfuric acid, phosphoric acid, nitric acid and hydrobromic acid. The acid may be selected from organic acids, such as from Ci-6 carboxylic acids. In some embodiments, the contact comprises dehydrating the dihydrofuran with an acid selected from the group consisting of formic acid. hydrochloric acid. sulfuric acid, phosphoric acid, nitric acid, hydrobromic acid, and Cl-6carboxylic acid. In some embodiments, the acid comprises formic acid.

In some embodiments, dihydrofuran is converted into HMFA at low pH. In some embodiments, pH can be acidic. In some embodiments, pH is neutral or basic. In some embodiments, pH is acidic and is from 0-6. pH can be 0, 1, 1.5, 2, 2.5, 3, 3.5, 4, 4.5, 5, 5.5, 6, 6.5. 7, 7.5, 8, or up to about 10. In some embodiments, the pH is 4. In some embodiments, the pH is 4.5. In some embodiments. the pH is 5. In some embodiments, the pH is selected from the group consisting of: 4, 4.5. and 5.

In some embodiments, pH is about 4 and temperature is about 63° C. In some embodiments, pH is about 4.5 and temperature is about 69° C. In some embodiments, pH is about 5 and temperature is about 72° C.

In some embodiments, the conversion (e.g. dehydration) of a dihydrofuran to HMFA yields from about 5%-100% HMFA. In some embodiments, from about 5-10%, 10-30%, 25-40%, 30-50%, 35-60%, 40-70%, 45-85%, 50-80%, 50-90%, 55-90%, or 60-100% is converted to HMFA. In some embodiments, conversion of a dihydrofuran to HMFA yields at least about or at most about 5%, 15%, 25%, 35%, 45%, 55%, 65%, 75%, 85%, 95%, or 100% of HMFA.

In some embodiments, HMFA can be further chemically and/or biocatalytically oxidized to FDCA. In some embodiments, the HMFA is purified before oxidation. In some embodiments, the purification comprises increasing HMFA in a composition. In some embodiments, the purification comprises increasing HMFA in a composition by at least about 1-fold, 5-fold, 10-fold, 20-fold, 30-fold, 40-fold, 50-fold, 100-fold, 150-fold, 200-fold, 300-fold, or 500-fold as compared to an otherwise comparable composition that lacks purification.

In some embodiments, the resulting FDCA can be used as a polymer building block. FDCA can comprise a low carbon footprint as compared to PET in plastic industry.

### Modified Microorganism

In some embodiments, any of the described glycoside hydrolases or motifs thereof can be produced in a host, such as a microorganism. A modified microorganism can refer to host cells that have been genetically modified by the cloning and transformation methods of the present disclosure. Thus, the term includes a host cell (e.g., bacteria, yeast cell, fungal cell, CHO, human cell, etc.) that has been genetically altered, modified, or engineered, such that it exhibits an altered, modified, or different genotype and/or phenotype (e.g., when the genetic modification affects coding nucleic acid sequences of the microorganism), as compared to the naturally-occurring organism from which it was derived.

In some cases, a microorganism is modified to produce a glycoside hydrolase. In some embodiments, the modified microorganism comprises and/or expresses any of the glycoside hydrolases provided herein. A modified microorganism can also comprise a polynucleotide that encodes for any of the glycoside hydrolases provided herein. Therefore, provided herein is also a modified microorganism that comprises and/or expresses a glycoside hydrolase and methods of making the same.

In some embodiments, a DNA sequence encoding a glycoside hydrolase or motif thereof is cloned into an expression vector and inserted into a production host such as a microbe, e.g., a bacterium. The protein can be isolated from the cell extract based on its physical and chemical properties, using techniques known in the art. In some embodiments, the sequences of the present disclosure may be introduced into a host cell using any of a variety of techniques, including transformation, transfection, transduction, viral infection, gene guns, or Ti-mediated gene transfer (see Christie, P.J., and Gordon, J.E., 2014 “The Agrobacterium Ti Plasmids” Microbiol SPectr. 2014; 2(6); 10.1128). Particular methods include calcium phosphate transfection. DEAE-Dextran mediated transfection, lipofection, or electroporation (Davis, L., Dibner. M., Battey. I., 1986 “Basic Methods in Molecular Biology”). Other methods of transformation include for example, lithium acetate transformation and electroporation See, e.g., Gietz et al., Nucleic Acids Res. 27:69-74 (1992); Ito et al., J. Bacterol. 153:163-168 (1983); and Becker and Guarente, Methods in Enzymology 194:182-187 (1991). Additionally, representative non-limiting techniques for isolating glycosyl hydrolase from a modified microorganism include centrifugation, electrophoresis, liquid chromatography, ion exchange chromatography, gel filtration chromatography, and/or affinity chromatography.

In some embodiments, a microorganism is modified to comprise and/or express an amino acid sequence that comprises from 80%-100% sequence identity to any one of SEQ ID NO: 1-116. In some embodiments, a microorganism is modified to comprise and/or express an amino acid sequence that comprises at least 10%, or at least 99.73% identity to an amino acid sequence selected from the group consisting of SEQ ID NOs. 1-116.

In some embodiments, a modified microorganism of the disclosure comprises an amino acid sequence that is at least 80% identical to an amino acid sequence of SEQ ID NO: 1. In some embodiments, the modified microorganism comprises a glycoside hydrolase polypeptide that comprises an amino acid sequence selected from the group consisting of SEQ ID NOs: 1-116. In some embodiments, the modified microorganism comprises a glycoside hydrolase polypeptide that SEQ ID NO: 19.

In some embodiments, the glycoside hydrolase polypeptide that is comprised or expressed by a modified microorganism further comprises a tag amino acid sequence. In some embodiments, the tag amino acid sequence is His6.

In some embodiments, a modified microorganism is a bacteria. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles. A bacteria can be any one of: E. Coli, Saccharomyces sp., Aspergillus sp., Pichia sp., Pseudomonas sp., or Bacillus sp. In some embodiments, the bacteria is E. Coli.

### EXAMPLES

The following examples are given for the purpose of illustrating various embodiments of the disclosure and are not meant to limit the present disclosure in any fashion. Changes therein and other uses which are encompassed within the spirit of the disclosure, as defined by the scope of the claims, will be recognized by those skilled in the art.

### Example 1: In-Vivo Production of Glycoside Hydrolases

Polynucleotides encoding amino acid sequences SEQ ID NO: 1 to SEQ ID NO: 116 were synthesized (Twist Bioscience) and inserted into the pARZ4 expression vector. The recombinant vectors were used in a heat shock method to transform E. coli NEBT7EL (New England Biolabs), thereby preparing recombinant microorganisms.

The transformed modified microorganism was inoculated with 1 ml TB-kanamycin medium, cultured by shaking at 37° C. overnight. 100 uL of culture was inoculated with 5 ml TB-kanamycin medium and grown for 2 hours at 37° C., followed by 25° C. for 1 hour, 400 RPM. The culture was induced with 1 mM IPTG and allowed to express for 20-24 hrs at 25° C., 400 RPM. Finally, culture was harvested by centrifugation at 2,200×g for 10-minutes. supematant discarded, and pellets stored at −20° C.

### Example 2: Purification of Glycoside Hydrolases

The microorganisms created in Example 1 were allowed to thaw from −20C storage. Once thawed, pellets were resuspended in a lysis buffer (2 mg/mL Lysozyme, 0.1 mg/mL DNAseI, 5% BugBuster® Protein Extraction Reagent, 20 mM PO₄ pH 7.5, 500 mM NaCl, and 20 mM Imidazole). Resuspended cells were disrupted by incubation at 5° C., shaking for 30 minutes at 200 RPM. The disrupted lysate was centrifuged at 2,200×g for 7 minutes. The obtained supematant was loaded onto a binding buffer-equilibrated Ni-NTA plate. The plate was centrifuged for 4 minutes at 100×g followed by two washes of 500 uL binding buffer (20 mM PO₄ pH7.5, 500 mM NaCl, 20 mM Imidazole) and two-minute centrifugation (500×g). The proteins were eluted with 150 uL elution buffer (20 mM PO4 pH 7.5, 500 mM NaCl, 500 mM Imidazole) followed by centrifugation for 2 minutes at 500×g. The recovered protein was desalted into a buffer solution for enzyme activity evaluation (50 mM acetate pH 5, 150 mM NaCl).

### Example 3: Measurement of Glycoside Hydrolase Activity with Keto-Sugar Substrate via UV Quantification Method

Purified enzyme from Example 2 was incubated in 50 mM of acetate buffer, pH 5 150 mM NaCl, 180 mM KDG (2-keto-3-deoxy-gluconate), for 3 hours at 74° C. Reaction mixture was then filtered with a 10K MWCO filter plate to remove protein from product, substrate, and other reaction components. Product formation was detected using a Biotek™ Synergy HTX Multi-Mode Microplate Reader. Product was measured at wavelength of 250 nm in a UV-transparent plate. Quantitation of product was achieved by calculation based off a KDG standard curve produced in the same reaction conditions.

### Example 4: Measure Glycoside Hydrolase Activity with Keto-Sugar Substrate via Triple Quadrupole (QQQ) Quantification Method

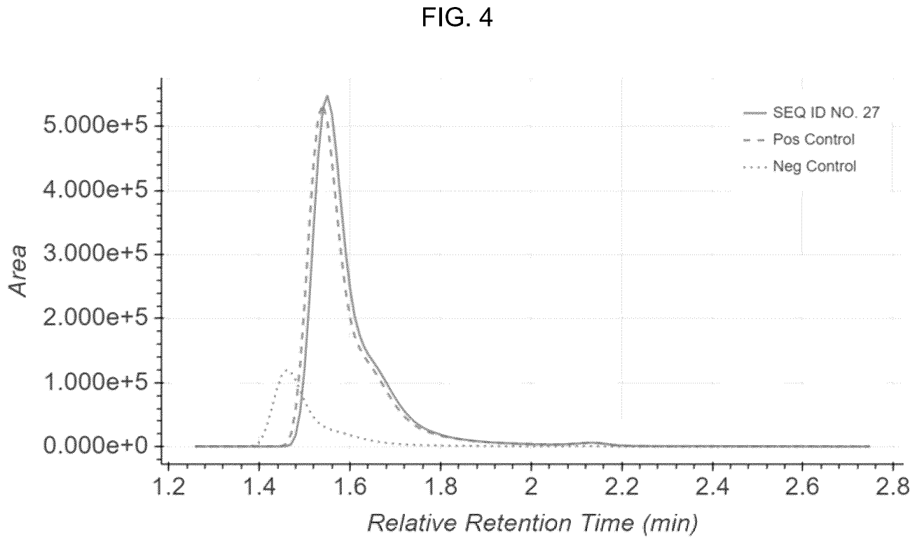

Purified enzyme from Example 2 was incubated in 50 mM of acetate buffer, pH 5 150 mM NaCl, 180 mM KDG (2-keto-3-deoxy-gluconate), for 3 hrs at 74° C. Reaction mixture was then filtered with a JOK MWCO filter plate to remove protein from product, substrate, and other reaction components. Product formation was detected using an Agilent G6470A triple Quad mass spectrometer. The LC components comprised of an Agilent 1290 multisampler at 10° C., 1290 infinity II pump, 1290 Flexpump, and a 1290 MTC running alternating Waters 100×2.1 mm HSS T3 1.8 um C-18 columns at 40° C. Received 384 well assay plates had 5 ul of 2 mM, 1 mM, 0.5 mM, 0.25 mM stock of HMFA in water added to select 50 ul wells for calibration (yielding a 200, 100, 50, and 25 uM final concentration) for the QQQ. Samples were run in an alternating fashion with one column performing separation and analysis where the second was re-equilibrating with the starting mobile phase. This gave a 2.5 min runtime per sample. Analysis was preformed using a Multi reaction monitoring (MRM) method on the QQQ with the following transition settings for the 13C labeled HMFA (m/z 147) and the HMFA (m/z 141), FIG. 3 and FIG. 4.

### Example 5: Using Rational Design Approach to Obtain or Improve Enzyme Activity for KDG Dehydration

Design of Natural Enzymes with Low-Level KDG Dehydration Activity to Higher Activity Levels.

To improve the activity of parent scaffolds that showed some initial KDG dehydration activity, computational enzyme design techniques were used to improve substrate interactions in the active site(s) of SEQ ID NOs: 1 and SEQ ID NO: 19, as summarized in Table 3. For these sequences, information about the crystal structure of the native protein provided an accurate picture of how substrate or transition state fits into the active site(s). Loops were identified in SEQ ID NOs: 1 and 19 amenable to flexible backbone designs. In total, using computational design, the enzyme efficiency was improved in five enzyme backbones introducing anywhere between 1 and 12 mutations to the parent sequence.

TABLE 3
Exemplary Glycoside Hydrolases

| SEQ | | Assay | Assay | | | Turnover (moles |
|---|---|---|---|---|---|---|
| ID | | Length | Length | [Enzyme] | [HMFA] | product/min)/ |
| NO | Favorable Test Conditions | (hrs.) | (mins) | uM | uM | (moles enzyme) |
| 17 | Na Acetate, pH 4-5, 150 mM NaCl, 63-74° | 3 | 180 | 1 | 8,000 | 44.44 |
| | C., 3 hrs, 1 uM enzyme, 180 mM KDG | | | | | |
| 24 | Na Acetate, pH 4-5, 150 mM NaCl, 63-74° | 3 | 180 | 1 | 7,000 | 38.89 |
| | C., 3 hrs, 1 uM enzyme, 180 mM KDG | | | | | |
| 25 | Na Acetate, pH 4-5, 150 mM NaCl, 63-74° | 3 | 180 | 1 | 4000 | 22.22 |
| | C., 3 hrs, 1 uM enzyme, 180 mM KDG | | | | | |
| 26 | Na Acetate, pH 4-5, 150 mM NaCl, 63-74° | 3 | 180 | 1 | 8000 | 44.44 |
| | C., 3 hrs, 1 uM enzyme, 180 mM KDG | | | | | |
| 27 | Na Acetate, pH 4-5, 150 mM NaCl, 63-74° | 3 | 180 | 1 | 6,000 | 33.33 |
| | C., 3 hrs, 1 uM enzyme, 180 mM KDG | | | | | |

### Site Saturation Mutagenesis of Glycoside Hydrolase SEQ ID NOs: 1 and 19.

To discover the amino acids at positions on SEQ ID NOs: 1 and 19 where point mutations increase the activity of KDG dehydration, saturation mutagenesis was performed around the active site at position no. 40, 41, 42, 88, 132, 133, 141, 147, 189, 211, 331, 332, 333, 334, 335, 336 and 352 of SEQ ID NO: 1 and at position nos. 41, 42, 87, 88, 91, 134, 149, 152, 193, 211, 219, 221, 225, 337, 338, 339, 352 and 356 of SEQ ID NO: 19. A total of 323 and 342 protein variants (19 point mutants per amino acid position) for SEQ ID NOs. 1 and 19, respectively, were examined for KDG dehydration activity.

Among the SEQ ID NO: 1 variants, 11 point mutations at 6 amino acid positions of SEQ ID NO: 1 resulted in up to 2-fold increase in KDG dehydration activity (SEQ ID NOs: 35, 37, 41, 42, 43, 48, 55, 62, 63, 64 and 68). The top 36 point mutations are: H42I, H42T, H42V, H42W, D88C, D88N, H132E, K133M, W141Y, H189A, H189V, V331K, G332E, G332M, G332V, S334A, S334C, S334D, S334E, S334G, S334I, S334K, S334M, S334N, S334Q, S334R, S334T, S334V, A335V, A335P, A335L, A335C, H352R and H352V. Flexible positions were discovered where multiple neutral or beneficial amino acid changes were found. For example, 20 amino acids were found at position nos. 332. 334 and 335 on SEQ ID NO: 1. Positions in the loop region of amino acid position nos. 331-336 in SEQ ID NO: 1 are more amenable to changes.

Among the SEQ ID NO: 19 variants, 8-point mutations at 3 amino acid positions resulted in up to 1.4-fold increases in KDG dehydration activity (SEQ ID NOs: 95, 96, 98, 99, 101, 102, 103 and 105). Point mutations that confer increased activity comprise: D41A, D41C, D41E, D41N, D41S, D41T, H87A, H87C, H87E, H87G, H87Q, H87R, H87S, L152M, L152N, W225Y, S337A, Y338V, H339A, H339N, W352F, Y356A, Y356C, Y356F, Y356H.

### Homology Modeling

Homology modeling was performed for SEQ ID NO: 4-18 and 22-34 using SWISS-MODEL homology modeling server available online. To perform the modeling the target sequence was uploaded in fasta format. A template search was performed using BLAST and HHblits. Searched templates were ranked according to Global Model Quality Estimate (GMQE) and Quaternary Structure Quality Estimate (QSQE). Top-ranked templates and alignments are compared to verify if they cover different regions of the target protein and represent alternate conformational states. Multiple templates were selected automatically and different models were built, accordingly. A template was selected (e.g. the first template) and used to build the model. The minimum percentage sequence identity used for template was 27%.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

1. A biocatalytic method of generating a dibydrofuran, the method comprising contacting a 2-keto-3-deoxygluconate (KDG) with a glycoside hydrolase, thereby generating the dihydrofuran, wherein the contacting comprises:
a. a pH from about 3 to about 7 as determined by pH meter, or
b. a temperature from 45° C. to 74° C.; or
c. both a. and b., thereby generating the dihydrofuran.

2. The biocatalytic method of claim 1, comprising a., wherein the pH is from about 4 to 5.

3. The biocatalytic method of claim 1, comprising b., wherein the temperature is from 70° C. to 74° C.

4. The biocatalytic method of claim 1, comprising a. and b., wherein the pH is from about 4 to 5 and the temperature is from about 62° C. to 72° C.

5. The biocatalytic method of claim 1, comprising a. and b., wherein the pH and the temperature are selected from the group consisting of:
a. pH about 4 and temperature about 63° C.;
b. pH about 4.5 and temperature about 69° C.; and
c. pH about 5 and temperature about 72° C.

6. The biocatalytic method of claim 5, comprising c.

7. The biocatalytic method of claim 1, wherein the KDG is from 100 mM to 2 M.

8. The biocatalytic method of claim 7, wherein the KDG is from 100 mM to 750 mM.

9. The biocatalytic method of claim 1, wherein the glycoside hydrolase comprises a protein with at least 80% sequence identity to a sequence selected from the group consisting of SEQ ID NO: 1-116.

10. The biocatalytic method of claim 9, wherein the sequence identity is at least 85%, 90%, 95%, 98%, 99%, or 100%.

11. The biocatalytic method of claim 1, wherein the glycoside hydrolase comprises
a first motif that binds the KDG, and
a second motif that comprises a catalytic residue, the catalytic residue comprising aspartic acid,
the first motif including at least two residues, a first residue comprising arginine and a second residue comprising tryptophan, phenylalanine, or tyrosine, and
the glycoside hydrolase is a homolog to SEQ ID NO: 1 or SEQ ID NO: 19 as determined by SWISS-MODEL modeling.

12. The biocatalytic method of claim 1, wherein the glycoside hydrolase comprises a protein with 100% sequence identity to SEQ ID NO: 27.

13. The biocatalytic method of claim 1, wherein the contacting is from 72 hours to 14 days.

14. (canceled)

15. (canceled)

16. The biocatalytic method of claim 1, further comprising
dehydrating the dihydrofuran to generate 5-hydroxymethyl-2-furoic acid (HMFA), wherein at least 40% yield of the HMFA is observed after the dehydrating.

17. The biocatalytic method of claim 16, wherein the dehydrating comprises contacting the dihydrofuran with an acid selected from the group consisting of: formic acid, hydrochloric acid, sulfuric acid, phosphoric acid, nitric acid, hydrobromic acid, and Ci-6 carboxylic acid.

18. (canceled)

19. The biocatalytic method of claim 16, further comprising
oxidizing the HMFA to generate 2,5-furandicarboxylic acid (FDCA).

20-50. (canceled)

51. An isolated unnatural glycoside hydrolase, comprising:
a first motif that binds 2-keto-3-deoxygluconate, the first motif including at least two residues, a first residue being arginine and a second residue being tryptophan, phenylalanine, or tyrosine; and
a second motif including a catalytic residue, the catalytic residue being aspartic acid or glutamic acid,
wherein the isolated unnatural glycoside hydrolase has at least 20% identity to SEQ ID NO: 1 or SEQ ID NO: 19.

52-57. (canceled)

58. The isolated unnatural glycoside hydrolase of claim 51, wherein the arginine of the first motif and the catalytic residue of the second motif are separated by about 70 residues.

59-65. (canceled)

66. A biocatalytic method of generating a dihydrofuran, comprising:
contacting a 2-keto-3-deoxygluconate (KDG) with a glycoside hydrolase, thereby generating the dihydrofuran, the contacting comprising
a. a pH from about 3 to about 7 as determined by pH meter;
b. a temperature from 45° C. to 74° C.; or
c. both a. and b., thereby generating the dihydrofuran,
wherein the glycoside hydrolase has at least 50% homology to SEQ ID NO: 1, and
wherein the glycoside hydrolase comprises, relative to SEQ ID NO: 1, D at residue 143, R at residue 213, and W at residue 217.

67. The biocatalytic method of claim 66, wherein residue 143 of the glycoside hydrolase is a catalytic residue and residue 213 and residue 217 are substrate binding residues.

68-77. (canceled)


SEQUENCE LISTING (116 sequences)

SEQ ID NO: 1 - protein, 373 aa, Bacillus subtilis
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTSAGFYDYYVSRERSTNDLHGAGAFILAMTELEPLFRSAGK

SEQ ID NO: 2 - protein, 377 aa, Persicivirga ulvanivorans
MNKSILLLVTLLSLYSCTDTEKTPLEEKDVFNEDYIKTSMIKALEWQEAHPIFAIHPTDWTNGAYYTGVARAHHTTKNMMYMAALKNQAVANNWQPYTRLYHADDVAISYSYLYVAENEKRRNFSDLEPTKKFLDTHLYEDNAWKAGTNRSKEDKTILWWWCDALFMAPPVINLYAKQSEQPEYLDEMHKYYMETYNRLYDKEEKLFARDSRFVWDGDDEDKKEPNGEKVFWSRGNGWVIGGLALLLEDMPEDYKHRDFYVNLYKEMASRILEIQPEDGLWRTSLLSPESYDHGEVSGSAFHTFALAWGINKGLIDKKYTPAVKKAWKAMANCQHDDGRVGWVQNIGAFPEPASKDSYQNFGTGAFLLAGSEILKMR

SEQ ID NO: 3 - protein, 361 aa, Clostridium acetobutylicum
MQKYSKLMADSIIAKNITLTDHWGYEYGLTLDGIAKVYEWTKDKKYLDFIIKTMDTFINEDGTINGYKLEEYNIDHLNNGKILITLFKETGKEKYRKALINLRKQIDNHPRTKENVFWHKNIYPHQIWLDGLYMGATFYAKYVKEFGEEKEFDDITHQFIITEKNLKDNKTGLLYHAYDESKTEPWSNSETGLSPHFWGRAMGWYVMALADTIEVLPKNHKDRNALIKILNNCVTALLKVQDNASKVWYQVLDEGERKGNYLEASGSSMIVYALLKGVRLGYLPESLKETAKEAYKGLINEFILETKDGLINLNKICYVAGLGGKDKRDGSFAYYISEPIVSNEPKGLGPFLLASYEYETL

SEQ ID NO: 4 - protein, 344 aa, Bacillus subtilis
MAQLIFDEEKVTSVIDRIVKRTFQMDFAWDWPGGVAFYGVAEAYEATENEEYINLLKTWVDEQLEDGLPPLSINGVSIGHTLLFLHKVTGDDVYLETAAEMAEYVLHKAPRFGEGILQHTVNAAEYVFPEQAWADTLMMAGLFMLRIGRVMEREDYFEDGLRQFHGHEDVLQDPVTNLYYHAWDNKAQNHLSGIYWGRANGWAALTMAKALPLIEVTHPSFMIIDGSLRDQLSALVRLQDESGLWHTILDDPDSYLEVSASAGIASALMSSGKLYTKYVQKSLAAILDAVEEDGRVSRVSAGTAVMKNAEGYKQVPYKRIQGWGQGLALTFLADVLKTKKRLYQ

SEQ ID NO: 5 - protein, 358 aa, Paenibacillus sp.
MSPIELATKACQSIMETYSPEQLPPAGRWHYHQGVFLVGLSQLWKFNGDAKFLDYIKGYVDHLVDANGNLLLERGELDSVQAGLLLMELDAVFPEYRYRAAADKLLRLLSTLNRTTEGGYWHKDRYPYQMWLDGLYMGGVFTMNYARQYNEPHLFDEVLFQEKLMRSHTKDEATGLYYHAWDESRTMPWADPVSGQSPEFWGRAIGWYALSLVEFLDLLPEHHSAREELAAALRELSEALMRYQDPATGLWHQVVDKGGRDDNWLETSCSSLFTYTLAKGVRLGYLDPSFMTYARQGYQGLVQSVYRNDKGNLVMPQICIGTGVGDYKHYVERQQCENDLHGVGALVMACVELGRSDG

SEQ ID NO: 6 - protein, 370 aa, unidentified
MDQSIAVKSPLTYAEALAKTIMNTYTVEELPPANRWHYHQGVFLCGVLGLWEATGQKRYFEYAKAYADLLIDDYGNLLFRRDELDAIQAGLILFPLYEHTKDERYVNAAKRLRGLYGTLNRTSEQGFWHKDGYPYQMWLDGLYMGGPFALKYANLKQEPELFDQVVLQESLMRKHTKDSKTGLFYHAWDEAKKMPWANEKTGCSPEFWARSIGWYVMSLADMIEELPKKHPNRHVWKKTLQDMIESICRYQDKETGLWYQIVDKGDRSDNWLESSGSCLYMYAIAKGINKGYLDRAYETTLLKAYQGLIQHKTETAEDGAFLVKDICVGTSAGFYDYYVSRERSTNDLHGAGAFILAMTELETLFRSAEK

SEQ ID NO: 7 - protein, 363 aa, unidentified
MVQTPSTPLEYAQAAVDTLRRTYPNPADLPPIRHFHYHQGVLLSGVHQTYLINNDESYLEYIRGWIESNLDDQGHVLNHDPVCLDDIQPGILLFPLYDHFGSSRYKTGLDQLADDLEAYPKNDEGGFWHNDGVPRQMWLDGLYMGGPMMVEYGTRFGRRNLVDEAVRQALLMQRKTRVPETGLWRHAYDPDRKEPWADPVTGLSPEYWGRSIGWVPMAVVNELELLPQSHPGREDLRNLVRDLLTAVCRYQGPDGRWWQVLDKVGQAGNWPENSCTCLFVAAICKAVDQGILDKTYLEVANKGYEGVITSLEWQGDDLLIGDICIGTGPGSYEEYCQRPTSINDMHGVGAFLLMCAGMERVRP

SEQ ID NO: 8 - protein, 358 aa, unidentified
MEKRPLDYAKASMDTMMRKWEAPKLPPEGRFHYHQGVFLSGMYKSYELCKEEKYFDYIKSWVDAVITEDGVIKQADMGQFDDMQPGILLYPLYHCTGDARYKKALDSLMAAIDCYPTTPEGGYFHKADLPEEMWLDGLYMEGPLRAQYGAEFHHPEYFDEVIFQALTMQKHTRDPKTGLLYHAWSYDRKVEWADKETGCSPEFWGRAMGWVPVALLDELDFIPADHPDRAKLEKMAVDLLKSLLAFQSEDGRWYQVVNRGDDPANWLENSCSCLYVAALCKAIRKGLLDASYLEYARKGYEGVIRSLTWEGDDLIIGNVCIGTGVGDYDHYIHRPVSANDLHGVGAFLLMCEECEQVF

SEQ ID NO: 9 - protein, 367 aa, Spirochaeta thermophila
MSHTSIDPLSVAREAAESLMHRYPDPAELPPAHRWHYHQGVFLLGMLKLWEETGEDRYLEYVKAYVDALVDEQGNLELATTELDAMQAGNLLFPLHERYGEARYKKAMDYILGLLEGFHTTPEGGFWHKEKYPNQQWLDGLYMAGPFSVHYASLFGRPELYDLFVKQALLIERHTRDPQTGLLRHAFDYSKQAPWADPVTGIAPEAWGRALGWFPAALLDILDHLPPSHTGYRVLEEMFRDLMGPIVRVQDRATGLWFQVLDKGHDPSNWLETSCSALFLYSLSKGIRTHRLPVDPYITHARKAFQGIVGKTSRDPEGYLHIHDICIGTGVGDYRHYLERPRSIDDLHGVGAFILGCVEFHRTRESI

SEQ ID NO: 10 - protein, 367 aa, Spirochaeta thermophila
MSHTPIDPLSVAREAAESLMHRYPDPAELPPAHRWHYHQGVFLFGMLKLWEETGEDRYLEYVKAYVDALVDEQGNLELATTELDAMQAGNLLFPLHERYGEARYKKAMDYILGLLEGFHTTPEGGFWHKEKYPNQQWLDGLYMAGPFSVHYASLFGRPELYDLFVKQALLIERHTRDPQTGLLRHAFDYSKQAPWADPVTGIAPEAWGRALGWFPAALLDILDHLPPSHTGYRVLEEMFRDLMGPIVRVQDRATGLWFQVLDKGHDPSNWLETSCSALFLYSLSKGIRTHRLPVDPYITHARKAFQGIVGKTSRDPEGYLHIHDICIGTGVGDYRHYLERPRSIDDLHGVGAFILGCVEFHRTRESI

SEQ ID NO: 11 - protein, 368 aa, Clostridium stercorarium
WRLDKPHTVIRPYNDNKLFGHWNYPLGVTLYGLAEAGRFIKDSSLVDYVTKHVELCTRFFDYAMWDRDRYGAASMHNLLSTLSTLDDCGSFGSLMLEVSGELNDDAYVRIADYIADFIRNRLDRLPDGAFYRVNPEHLLMDQTLWADDLYMSVPFLCRYYKLTGRQEFIDDAAKQLVLYHKYLYRPDKKIMSHVYDVRHRKATEVSWGRGNGWVAFSYSELLRFLPENHELREELIRNFNDLCEGYLALQDEKGMWHQVLTDPDSYPEASCTSMFACAFARGVRNSWLNEPEKYIEAVEKAWKGLCSEAIDLHGNVYGICRGSGFSFSEDYYKNDLGWLLNDTHGTGIVLLAGVEYGKLLEWLDNGGI

SEQ ID NO: 12 - protein, 366 aa, unidentified
MKTSFERSLFVFLLIVLGIAEVRAQSDYFARWPAGASPREVGRRVAENFLARPFDFETNPRRAYVIYPEVCTWYGALRVARLTGDRELQRRLIAKFDRFLTPEGAKRISPEAHVDFRVFGVVPLEIYMQTKDRRYLEIGKSLADRQWEKTTPDGITAEARYWIDDMYMITAVQVQAYRATGDARYLERAAQTMVAYLDKLQQPNGLFYHAPDSPYFWSRGNGWVAAGMTELLISLPRNHPKRERIMEGYRKMMATLLKYQGEDGLWRQLIDHPEAWAETSGTGMFTFAMVTGVKRGWLDAKTYGPAARKAWLGLVSRLDEKANLRDVCVGTNKGYSVQYYLDRPRSTGDLHGQAPLLWSAAALLEH

SEQ ID NO: 13 - protein, 366 aa, Thermotoga thermarum
MKYEKVERFINEYLSSYKPYKGRWCYEDGCVLKGSWDLYRVTGNQRYLDFVSSYFENFIDEQGNVKGYNLENYALDDICPASVLIDFYEIEKKEKYRKALDLFYKQIISHPRTKIGNFWHKKIYPNQIWLDGLYMVMPFYTKYAKKFLNFEEALKDIVNQFLNVRKYMWDEKKKLYYHGYDESRVQEWADKITGLSKSFWARAQGWFMMALVDVLDAITDIDKSQYFNSLADIFVEAAYGLINYQDEETGMWYQVIDQVRPDNYLETSATLMFAYSFLKGFRLGLLGSEFKDRGLKAFDGTVKKYLVEKDGKLSLHGICSVAGLGWYENRYRDGSYKYYLSEKVVSDDPKGVGPLMMAYSELLNIS

SEQ ID NO: 14 - protein, 376 aa, Caldalkalibacillus thermarum
MSHNNQNDVTDHHSPLFWGERACETLMRSYSPPQLPPENQWHYHQGVYLYGMYRLWEQTGKEEYYNYFKAYVDHLVDEYGNFLFRRDELDAIQAGLLLFPLYRLTKHSKYKLAAQKLRNLFNTLNKTAEGGFWHKDKYPYQMWLDGLYMAGPFACIYALMFNEPELIDMVLKQERLMRSHMKDPQTGLYYHGWDESKQTAWSDPVTGCSPEVWGRSLGWYGMALADIIEVLPENHSGRKELIETLRAYLESVIRYQDEETGMWYQVIDKGSEPDNWLETSASCLFIYTIAKAVNLGVAEQKLINAADKGFKGVIEQKVKTREDGLLSLEDICIGTSVGVYDYYVNRERSANDLHGVGAFILACVELHKNKAGKRGN

SEQ ID NO: 15 - protein, 373 aa, unidentified
MGVSAQTPAHPQKVVPVDRNAAGDAPDDPGPLAANLSSAIQPKAISAAMKKVADWELTVAEPSFNQQWTFAALYDGLLAASATTGDAKYRNAVVRMSQRYDWQLLDNRFPHADDQALGQAYLDLYLRDRKPVRLANTKAIMDRLVVRPDDPNKLLWWWCDALFMSPPVLLRTYAITHDQKYLDYMDHEWWLTSAKLYDPEEHLYFRDDRYLNQKQENGRKLFWSRGNGWVMGGLVNVLEYLPADYPSRAKYVEQFRQMAAALAAIQSADGLWRSGLLDPDAYELPEVSGSAFVTYGIAYGINHNILDRAKYQPVVEKSWKGMLSHIYADGRLGSIQPIDGQPGKFKPSASYVYGVGGFLLAGSEMHKLAAAKH

SEQ ID NO: 16 - protein, 410 aa, unidentified
MSRFGWKYGAVIPVLLLSFGLQEIHAQQVSYEHATAKQLAGIAKDNSRHFGDDPDHPGPLAKDLSYGIAPAPVGKAMRKVADWQLERSQPYFDRIWTWSVLYSGFMAASESLNDPKYRDAMMAMGAKFDWRLRSHLPNADDQSIGQTYLELYLLKKDPAMMQPTRDELDAVLAAPRMSTIPGKELPWWWCDALFMAPPVWARMYAATGDRKYLSYLDEEWWKTSDLLYDTTEHLYARDASYLTRKEANGQKMFWGRGNGWVMGGIARTLEYLPKDDAARPKYVKQLQEMSARVASLQGKDGMWRSGLLDQSNYALPEMSGSALMTYALAWGVNNGILDRKVYRPVIQKAWAGMLHHVYADGRLGCIQQTGAEPAPFKASASYTYGVGAFLLAGSEIRRMEGNSSVGKRVH

SEQ ID NO: 17 - protein, 366 aa, unidentified
MQCKSMCILAITTSLLIAPFALSAGQIEAFQQWPEGAAPAQVGKRVAENFLKRSFDFETNQRRRYIIYPEACTWYGALTLARLTNDKDLADRLIKRFDFLLTPEGSKRISPEAHVDYRVFGIVPLEIYIQTKDKRYLDVGLGLADAQWEKTTDDGITAEARYWIDDMYMITAVQTQAYRATGKAKYIDRAALAMASYLDRLQQPNGLFYHALDVPFFWGRGNGWVAAGMAELLRSLPEDHPKRPRIMEGYKKMMDALLRYQDPNGMWRQLIDRPESWPETSSTGMFTFAMVTGVKNGWLDKETYAPAARKAWLALVGFIDEDANVRQVCVGTNKLNNYQYYIDRPRSTGDLHGQAPVLWTASALLR

SEQ ID NO: 18 - protein, 350 aa, unidentified
MASSIDRQAVQSCLERLASHSGEELLSKPPHWGDAIMCDGLLYAARALRIEAPVRRAIKWFEPKLAAGPDLERWFWFWSAEALPALGLYQATGKSDYLEYARAIVDTVEHKTPHTADGVPIPHPPEPIVWIDVVYFVAPAMALLGRLTRDEAMVGRALDQVLLHAKYLLDPVTDLFWHVANAEKRTHSPCLWARGNSWFSIACSQVLREIKTAGLEGRFGGKAEDAALDLARQMNAISALQDQASGLWHTVLEQPTSYLESSASAGFALGVGRALQLDLDRLNSFRARRTYERAISAICTKINSAGEFTGVSQQTPPGDFAHYQSIEVGTAPFGSGVCMMALAEALEQAP

SEQ ID NO: 19 - protein, 377 aa, Bacillus sp.
MWQQAIGDALGITARNLKKFGDRFPHVSDGSNKYVLNDNTDWTDGFWSGILWLCYEYTGDEQYREGAVRTVASFRERLDRFENLDHHDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYYYLEALLRLERGVTGYWYERGR

SEQ ID NO: 20 - protein, 398 aa, Streptococcus agalactiae
MMKIKPVKVESIENPKRFLNSRLLTKIEVEEAIEKALKQLYINIDYFGEEYPTPATFNNIYKVMDNTEWTNGFWTGCLWLAYEYNQDKKLKNIAHKNVLSFLNRINNRIALDHHDLGFLYTPSCTAEYRINGDVKALEATIKAADKLMERYQEKGGFIQAWGELGYKEHYRLIIDCLLNIQLLFFAYEQTGDEKYRQVAVNHFYASANNVVRDDSSAFHTFYFDPETGEPLKGVTRQGYSDESSWARGQAWGIYGIPLSYRKMKDYQQIILFKGMTNYFLNRLPEDKVSYWDLIFTDGSGQPRDTSATATAVCGIHEMLKYLPEVDPDKETYKYAMHTMLRSLIEQYSNNELIAGRPLLLHGVYSWHSGKGVDEGNIWGDYYYLEALIRFYKDWELYW

SEQ ID NO: 21 - protein, 402 aa, Pedobacter heparinus
MKSLLSAFVATIALIGSANGMTVTKGNGDDWLKKSTKTAVIQLTRAAQTYTPGMNPRSVNPDGTVRLAPPRDWTTGFFPGTLWYGYELSGDKNLAAEAKRFTLALDTIQYVKDTHDLGFMLYCSYGNAYRVTGDKIYLKPLENGAANLYARFNKKVGAIRSWDFGHWQFPVIIDNLMNLEYLYWAGKEFNKPEWFDAAKTHAVTTMKNHFRKDYSSYHVISYDTLSGKVLQRETHQGLTNESAWARGQAWGLYGYTMSYKDTKDKKFIEHAEHIAAFIMNHPAMPADKIPLWDFDVHNRDRSPRDASAAAVIASALLDLSTQVKDGQKYFKFAEDILKTLSSDEYLAKPGENQFFILKHSVGALLYNSEIDTPLNYADYYYLEALKRYAEIKKIDLKTINQS

SEQ ID NO: 22 - protein, 401 aa, Caldicellulosiruptor kronotskyensis
MNGGTKMLKIKDEGIKDRFKFSNSPLVSKAFVGEVLKLVLSKLDRMIEKFGDKFPSPASKALKYDVVGNIYWTSSFYTGMLWLAYEVTGEEKYKDLAKRHLDSFEKRLKSRIHIDTHDLGFLYTLSCVACYKVTGDERAKEVAIEAAKQLCTRYWEKPGIIQAWGAMDDPVQKGRIIIDCLMNLPLLYWASTQTGDKKFFDIARMHAQMTAENIVREDASTFHTFYFDVETGKPLYGSTHQGYSDSSCWARGQAWGIYGFVLSYKYTRDWIFIDIAKKLLNYFLNRLPEDCVPYWDLVFTEGEQPRDSSAAAICACGILEMLKFLPLCDEYREYYENAAKNIVFSLSTKYLASQDLDSDGLLLHGVYNKPKNEGVDEHTIWGDYFFFEALVRFLQYWHEYW

SEQ ID NO: 23 - protein, 377 aa, unidentified
MTLRPEEISKFQEALQFAEKQVAALVEKHPDFFPIYTVNGRWYHEGELWTDWTGGFLAGMMWQFYLRARDPKWWELATHYSRLLEHRKHDRNVHDLGFIFLNTYLPWYELTGDQALLDVLITAGRTLAMRFQPKGKYLCSFIGPHSLFIDIMMNVPLVIFAALKTGDQELLEIGLAHCRTTRDYLVRPDGSTAHEGLFDVETGQFLGQSTHQGLSPESCWARGLAWSMYGFSKVYALTNNPEFLEVAERNAEYWITHLPEDKIPYWDFHADLSQPPPWGRQKESSAAAIAASGLWDLAKQTRSPERGLVYRNIALVMLSALCDPEYLAIETPGWEGILKHGVYHTRKNLGVDESVMWGDYFFVEGLTKVLTAEGNNS

SEQ ID NO: 24 - protein, 361 aa, Sulfolobus islandicus
MKFRDALEELKLNIINLVYEYKEKSEFPIYLDEKSGAWKTSNGRHWMEGFLVGILWNLYDFFRDEKIKEVAVEYTLKLLKIEAPITHDIGFRMYYSAVKGWENMYANQLKKNIITYANILASIFREDLGFIPVGDDFVTYFPGEDKKIAKSELIIDTMMASLPFLAWVSSNFDINYKNIIILHAHKTFDLLVRSDGSTYQAAYINDNLEIFRHTHQGYSNESCWARGQTWGIYGYTILYSYLKNEIFLKIARKLASFFVKNIPRDKIMYYDFQDTTEKDSSVNVIGASSLINLYRETYEVDYLRTAVTLVRAVINNREYFREGGLKHTRYRKDEGKDSETMFGDYYLSEALINLIKMGIEE

SEQ ID NO: 25 - protein, 362 aa, unidentified
MKFRTALEELKSNIVNLAYEYREKNEFPIYTDERKGSWTTSNGQHWMEGFLVGILWNLYSFFRDEKIKKLALEYNSKLLEIEGPLSHDIGFRVYYSAVKSWENTYSNDLKKKIITYADKLASLFREDLGFIPVGEEFVTYFPGENKEIAKSELIIDTMMASLPFLSWVLTNFDINYYKNIIIMHAHKTFDLLVRNDGSTYQAAYIDDNLEVFKHTHQGYSNESCWSRGQTWGIYGYSLIYSYLKKEIFLKIARKLASFLMKNIPEDKIMYYDFQDTTEKDSSSNVIGASALINLYRETYEIEYLRNAVELIRAIINNKEYFREGGLKHTRYRKDQGRDSETMFGDYYLSEVLINLIKMGIEE

SEQ ID NO: 26 - protein, 361 aa, Sulfolobus islandicus
MKFRDALEELKLNIINLAYEYKEKSEFPIYLDEKSGAWKTSNGRHWMEGFLIGTLWNLYDFFRDEKIKELAVEYTLKLLKIEAPITHDIGFRIYYSAVKGWENMYSNQLKKNIVTYANILASIFREDLGFIPVGEDFVTYFPGEDKKIAKSELIIDTMMASLPFLAWVSSNFDMNYKNIIILHAHKTFDLLVRNDGSTYQAAYINDNLEIFKHTHQGYSNESCWARGQTWGIYGYIILYSYLKNEIFLKIARKLALFFMKNIPGDKIMYYDFQDTTEKDSSVNVIGASSLINLYRETYEVDYLRTAVTLVRAVINNREYFREGGLKHTRYRKDEGKDSETVFGDYYLSEALINLIKMGIEE

SEQ ID NO: 27 - protein, 364 aa, unidentified
MDFAECLKEISTNAWNLYENYGNEFPLYRAHGSEKWVTSKGEHWMEGFYAGLLYKAYEFTKEDKFLGAGLKITEQIISSRVPITHDIGFKFYYSAVAGYNLTGDNKFKSVALKMASELADTFNPNLGAIPLGREFAAYFPGEPPSQANNEFIIDAMMASIPLLLWAWTMTSEPKYYYIAYIHSMRTFDLMVRSDFSSYQAAFLDVSSLSLNKHTHQGSSNESTWSRGQAWAIYGFLEMYKHTHLDFFKNVSLRMADYFLNHLPQDYIPPYDFSESTNTGAARDTSAAAIAGSAITQLSLLTGNNHLLEKSVEIARALTSPDYFSSGRLLHSRYREDQGKDAELIFGDYYLAELLLELWRYGVKK

SEQ ID NO: 28 - protein, 354 aa, unidentified
MELSELIINKVENCLNLNEFPNITKNGKWTTTKDGFWTGGFWIGLLWLIYIITENKKYLKSAYRWANNLEKRCKDKTFDLGFLFYPSFVLGYEITNDEYFRRVALTSASTMTTLFNDKVGFIYNEIKDNNSITGRTIIDVMMNIPLLWWAYRETGDPTFLKVASTHSLRTAEELVREDGSTIQVLDFDIKTGKIFRKHTTQGYNSNSCWSRGQAWALYGFALAYKATKNKLIKRVLQRVVNYYVSNLPSDFVPFWDFRDPNIPNCPKDTSASAIACSGMMETRDHMDVCTRAIGSLTEKYVKTYGDGILSHGCFYKPAGLGIGESLIWGDYYFVEALLKLEGNEIVKNLYTYRL

SEQ ID NO: 29 - protein, 395 aa, unidentified
MLKIKDEGIKDKLKFSRSPLVSKAFVEEVLKLVLSKLDRMIEKFGDKFPSPASKDLKYDVVDNIYWTSSFYTGMLWLAYELTGKEKYKDLAKRHLDSFEKRLKSKSYIDTHDLGFLYTLSCVACYKVTGDEGAKNIAIEAAKQLCTRYWEKPGIIQAWGAMDDPAQKGRIIIDCLMNLPLLYWASIQTGDKKFFEIAHKHAKRTAENIVREDASTFHTFYFDVETGKPLYGSTHQGYSDSSCWARGQAWGIYGFVLSYKYTRDWIFMDIAKKLLNYFLNRLPEDYVPYWDLVFTEGEQPRDSSAAAICICGILEMLKFIPLCDEYREYYENAVKNMVFSLSTKYLASQDLDSDGLLLHGVYNKPKNEGVDEHTIWGDYFFFESLMRLVKDWNTYW

SEQ ID NO: 30 - protein, 375 aa, unidentified
MKLKFWMRNKIFWALIFTLGLALVCIEGAGSSQASSDRDPVVAGQRAAHWVMSRNWFYNDPINLIIGPADSYYQTVCARYGVLIFADATGNKDLLLKVQQAYQPFLTGKRHPNTGHVDLNVFGIVPFELYRQTSDSRYLPLAKMLADDEFKGSRADGLSDYTRFWVDDMYMVGSLQVQAYKSLKDPVYLDRAAVQLLAYIDKLQQPNGLFYHTPGTPFFWARGNGWASAAMTEWLLSADDAHPKKAEIFKAYKKMMDALIELQDENGLWHQLLDEPESYIESSGSGMFIFALATGVRKGWLPDQPFRSALEKAWNALSEYLDAEGRVQEVCVGTAALPSKSYYMTRPSKSGDMHGQAGFLWASTAIYLLEKEKSQ

SEQ ID NO: 31 - protein, 364 aa, unidentified
MKTINQCFAILLFLALNITVTAQDDAPGRIPQYDVPYEYPTVNGIVDVLNNVRGYYESTSLQIIIDAKTGDEITDFSKVNKNAEVSGGFSNSWSYTHGVVLSAFDYIDDVIGDPAFFANNTRFYDLVIETLPYFQKNREKFGDEASNGWGRTPNFHALDDCGSIGAAMIKTYLKDKNKNYLELINITADYISNKQFRLEDGTLARHRPQYQSIWSDDIYMSVPFLANMGVLTGDNTYFDDAVKQILQMADRLYIPHIELFDHGWSVPSGDYDPRFYWGRANGWTLMAMAELLSVLPEDYPGRDEVLHLYRSMIRSLAGLQDGSGFWHNMLDKTNTYLETSCTAMFTFAVAKGINEGWMKPMIRY

SEQ ID NO: 32 - protein, 353 aa, unidentified
MDWKNILSYAIKKVDRSIKLTDFPHITKNGKWVTTKDGYWTGGFWIGLLWLAYKITNNQKYKKMAYSWVKRLEGRRFDKTFDLGFLFYPSFVLGYKLTGDEYLGQVSLDAAKTLSTLFHKKSKFIYCVINVDGRRIGRTIIDVMMNLPLLWWAFEETDNSVYYDVAYEHSIQTIKEFIRKDYSTIHVIDFDLDTGNIIRKITVQGYSNESCWSRGQAWAIYGFALAYKTAKDSIFLETAEKLADYFIKYLPEDYVPYWDFNDPKKSVKDSSAAAIASSGLLTLSNLSQQEKFKEIAVRILNSLSKNYLANENEDGILKHGCFHKPAGIGVDECLIWGDYYFMEALMKIRGELE

SEQ ID NO: 33 - protein, 432 aa, unidentified
MINTKKIAFASFLLLWIFTTIALFPQTKINGSEEQIVCVDDTTNSKSNLKDLIRLVAYNKIEEVSNGEYIKGTWEEVKNAKKSKITYWSYPTGVSLYALQRVYKILNDEKIINYVNNDLLISANVYEYLRWQKYKFGTIYNTQNLGKLWRLNMLDDCGVMGAALLESLMNFNLEPTTEIKQLVEIISNFVINVQARLYDKTFWRPKSPNSPSIWADDLFMSLPFLIKWSQYSKDKSYLTDAAYQVVSFASYLQDSIDGVWFHGYYVEKKERTCCKWGRGNGWVAIAITELLSALPENHKFYNQVLDIYKRQMSGIKKYQDDDGLWNQVLDHPELSFGTETSCSAQFAYAIARGINRGWLVKKNYINVINHALAGLKSRITKNGAINKVCKSTSLGDSLEYYNSRPIRDNDYHGNGLMLMALTEIYYLQKEYK

SEQ ID NO: 34 - protein, 449 aa, unidentified
MRYFIHYFTILIVFATYSVNLNSQLTDVAVPLHELMPDYKFDYGNIDTNYIKNKIFDILDYLDRATPAKLLDSKTGKEFKDFKLIDSNTEFVKGDFRLISYEWGVVYSALIYAGNCFNENKFKEYTKTRINFIKNVADYYKNIDSKNIKNTQIKSLLYPHALDDAGSMCAAMIKSKIYLNLEGIDWLIDNSIDFVLTKEYRLPDGTLARNRPHKNTIWLDDLYMSVPALAWMGKLKNDKKYFDEAIKQVISYYNILFDKEKSLFIHGKLLDYDFRPGFHWGRANGWAILAITELLDVMPENHPEYITILNILKQHIKGIVNYQSGDGLWHQLLDRNDTYLETSASAIFTYAIAKAINKGYLSAKAYAPCVIAAWNALSSKINKNGQVEGTCVGTGLGFDPVFYYYRPVSVYAAHGYGPVILAATEIYNLIKNFDIRIVEKAIVVYDKNK

SEQ ID NO: 35 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTSCGFYDYYVSRERSTNDLHGAGAFILAMTELEPLFRSAGK

SEQ ID NO: 36 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTSLGFYDYYVSRERSTNDLHGAGAFILAMTELEPLFRSAGK

SEQ ID NO: 37 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTSPGFYDYYVSRERSTNDLHGAGAFILAMTELEPLFRSAGK

SEQ ID NO: 38 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTSVGFYDYYVSRERSTNDLHGAGAFILAMTELEPLFRSAGK

SEQ ID NO: 39 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELCAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTSAGFYDYYVSRERSTNDLHGAGAFILAMTELEPLFRSAGK

SEQ ID NO: 40 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELNAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTSAGFYDYYVSRERSTNDLHGAGAFILAMTELEPLFRSAGK

SEQ ID NO: 41 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVETSAGFYDYYVSRERSTNDLHGAGAFILAMTELEPLFRSAGK

SEQ ID NO: 42 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVMTSAGFYDYYVSRERSTNDLHGAGAFILAMTELEPLFRSAGK

SEQ ID NO: 43 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVTTSAGFYDYYVSRERSTNDLHGAGAFILAMTELEPLFRSAGK

SEQ ID NO: 44 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWEKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTSAGFYDYYVSRERSTNDLHGAGAFILAMTELEPLFRSAGK

SEQ ID NO: 45 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYAAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTSAGFYDYYVSRERSTNDLHGAGAFILAMTELEPLFRSAGK

SEQ ID NO: 46 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYVAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTSAGFYDYYVSRERSTNDLHGAGAFILAMTELEPLFRSAGK

SEQ ID NO: 47 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTSAGFYDYYVSRERSTNDLRGAGAFILAMTELEPLFRSAGK

SEQ ID NO: 48 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTSAGFYDYYVSRERSTNDLVGAGAFILAMTELEPLFRSAGK

SEQ ID NO: 49 - protein, 373 aa, synthetic construct
MGSMDQSIAVKSPLTYAEALANTIMNTYTVEELPPANRWHYIQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTSAGFYDYYVSRERSTNDLHGAGAFILAMTELEPLFRSAGK

SEQ ID NO: 50 - protein, 373 aa, synthetic construct
MGSMDQSIAVKSPLTYAEALANTIMNTYTVEELPPANRWHYLQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTSAGFYDYYVSRERSTNDLHGAGAFILAMTELEPLFRSAGK

SEQ ID NO: 51 - protein, 373 aa, synthetic construct
MGSMDQSIAVKSPLTYAEALANTIMNTYTVEELPPANRWHYTQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTSAGFYDYYVSRERSTNDLHGAGAFILAMTELEPLFRSAGK

SEQ ID NO: 52 - protein, 373 aa, synthetic construct
MGSMDQSIAVKSPLTYAEALANTIMNTYTVEELPPANRWHYVQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTSAGFYDYYVSRERSTNDLHGAGAFILAMTELEPLFRSAGK

SEQ ID NO: 53 - protein, 373 aa, synthetic construct
MGSMDQSIAVKSPLTYAEALANTIMNTYTVEELPPANRWHYWQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTSAGFYDYYVSRERSTNDLHGAGAFILAMTELEPLFRSAGK

SEQ ID NO: 54 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHMDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTSAGFYDYYVSRERSTNDLHGAGAFILAMTELEPLFRSAGK

SEQ ID NO: 55 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTAAGFYDYYVSRERSTNDLHGAGAFILAMTELEPLFRSAGK

SEQ ID NO: 56 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTCAGFYDYYVSRERSTNDLHGAGAFILAMTELEPLFRSAGK

SEQ ID NO: 57 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTDAGFYDYYVSRERSTNDLHGAGAFILAMTELEPLFRSAGK

SEQ ID NO: 58 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTEAGFYDYYVSRERSTNDLHGAGAFILAMTELEPLFRSAGK

SEQ ID NO: 59 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTGAGFYDYYVSRERSTNDLHGAGAFILAMTELEPLFRSAGK

SEQ ID NO: 60 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTIAGFYDYYVSRERSTNDLHGAGAFILAMTELEPLFRSAGK

SEQ ID NO: 61 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTKAGFYDYYVSRERSTNDLHGAGAFILAMTELEPLFRSAGK

SEQ ID NO: 62 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTMAGFYDYYVSRERSTNDLHGAGAFILAMTELEPLFRSAGK

SEQ ID NO: 63 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTNAGFYDYYVSRERSTNDLHGAGAFILAMTELEPLFRSAGK

SEQ ID NO: 64 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTQAGFYDYYVSRERSTNDLHGAGAFILAMTELEPLFRSAGK

SEQ ID NO: 65 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTRAGFYDYYVSRERSTNDLHGAGAFILAMTELEPLFRSAGK

SEQ ID NO: 66 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTTAGFYDYYVSRERSTNDLHGAGAFILAMTELEPLFRSAGK

SEQ ID NO: 67 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTVAGFYDYYVSRERSTNDLHGAGAFILAMTELEPLFRSAGK

SEQ ID NO: 68 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICKGTAAGFYDYYVSRERSTNDLHGAGAFILAMTELEPLFRSAGK

SEQ ID NO: 69 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMYLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTSAGFYDYYVSRERSTNDLHGAGAFILAMTELEPLFRSAGK

SEQ ID NO: 70 - protein, 373 aa, synthetic construct
MGSMDQSIAVKSPLTYAEALANTIMNTYTVEELPPANRWHYKQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICTDTTAGFYDYYVSRERSTNDLAGAGAFILAMTELEPLFRSAGK

SEQ ID NO: 71 - protein, 373 aa, synthetic construct
MGSMDQSIAVKSPLTYAEALANTIMNTYTVEELPPANRWHYKQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICSDTTAGFYDYYVSRERSTNDLAGAGAFILAMTELEPLFRSAGK

SEQ ID NO: 72 - protein, 373 aa, synthetic construct
MGSMDQSIAVKSPLTYAEALANTIMNTYTVEELPPANRWHYKQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICTETTAGFYDYYVSRERSTNDLAGAGAFILAMTELEPLFRSAGK

SEQ ID NO: 73 - protein, 373 aa, synthetic construct
MGSMDQSIAVKSPLTYAEALANTIMNTYTVEELPPANRWWYAQGVFLCGVLRLWEATGEKRYFEYAKAYADLLIDDNGNLLFRRDALDAIQAGLILFPLYEQTKDERYVKAAKRLRSLYGTLNRTSEGGFWHMDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVGTTAGFYDYYVSRERSTNDLHGAGAFILAMTELEPLFRSAGK

SEQ ID NO: 74 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVQTSAGFYDYYVSRERSTNDLHGAGAFILAMTELEPLFRSAGK

SEQ ID NO: 75 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDEMDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVSTSAGFYDYYVSRERSTNDLHGAGAFILAMTELEPLFRSAGK

SEQ ID NO: 76 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVETSPGFYDYYVSRERSTNDLHGAGAFILAMTELEPLFRSAGK

SEQ ID NO: 77 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVQTSPGFYDYYVSRERSTNDLHGAGAFILAMTELEPLFRSAGK

SEQ ID NO: 78 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVETSVGFYDYYVSRERSTNDLHGAGAFILAMTELEPLFRSAGK

SEQ ID NO: 79 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVQTSVGFYDYYVSRERSTNDLHGAGAFILAMTELEPLFRSAGK

SEQ ID NO: 80 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVETTAGFYDYYVSRERSTNDLHGAGAFILAMTELEPLFRSAGK

SEQ ID NO: 81 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVSTSPGFYDYYVSRERSTNDLHGAGAFILAMTELEPLFRSAGK

SEQ ID NO: 82 - protein, 373 aa, synthetic construct
MGSMDQSIAVKSPLTYAEALANTIMNTYTVEELPPANRWHYHQGVFLCGVLRLWEATGEKRYFEYAKAYADLLIDDNGNLLFRRDELDAIQAGLILFPLYEQTKDERYVKAAKRLRSLYGTLNRTSEGGFWHKDGYPYQMWLDGLYMGGPFALKYANLKQETELFDQVVLQESLMRKHTKDAKTGLFYHAWDEAKKMPWANEETGCSPEFWARSIGWYVMSLADMIEELPKKHPNRHVWKNTLQDMIKSICRYQDKETGLWYQIVDKGDRSDNWLESSGSCLYMYAIAKGINKGYLDRAYETTLLKAYQGLIQHKTETSEDGAFLVKDICVQTTVGFYDYYVSRERSTNDLHGAGAFILAMTELEPLFRSAGK

SEQ ID NO: 83 - protein, 373 aa, synthetic construct
MGSMDQSIAVKSPLEYAEKLAQTIMNTYRPEELPPANRWHYHQGVFLCGLLRLWEKTGDKRYFDYAKAYADHLIDDNGNLYFARDELDAIQAGLILFTLYEETKDERYVIAAKKLRSLYDTLNRTSEGGFWHKDKYPYQMWLDGLYMGGPFALKYANMKQEEELFDQVVLQEKLMRKHTKDEKTGLFYHAWDESKKQPWADPETGCSPEFWARSIGWYVMALADMIEELPENHPNREVWTKTLQEMIDAICRYQDEETGLWYQIVDKGDRPDNWLESSGSCLFMYAIAKGINMGYLDPSYLEVLLKAYQGLIQHKTETSKDGAFLLSDICVETSVGDYDYYVSRERSTNDLHGAGAFILAMTELEDLFRSSGK

SEQ ID NO: 84 - protein, 373 aa, synthetic construct
MGSMDQSIAVKSPLEYAEKLAQTIMNTYRPEELPPANRWHYHQGVFLCGLLRLWEKTGDKRYFDYAKAYADHLIDDNGNLYFARDELDAIQAGLILFTLYEETKDERYVIAAKKLRSLYDTLNRTSEGGFWHKDKYPYQMWLDGLYMGGPFALKYANMKQEEELFDQVVLQEKLMRKHTKDEKTGLFYHAWDESKKQPWADPETGCSPEFWARSIGWYVMALADMIEELPENHPNREVWTKTLQEMIDAICRYQDEETGLWYQIVDKGDRPDNWLESSGSCLFMYAIAKGINMGYLDPSYLEVLLKAYQGLIQHKTETSKDGAFLLSDICVQTSVGDYDYYVSRERSTNDLHGAGAFILAMTELEDLFRSSGK

SEQ ID NO: 85 - protein, 373 aa, synthetic construct
MGSMDQSIAVKSPLEYAEKLAQTIMNTYRPEELPPANRWHYHQGVFLCGLLRLWEKTGDKRYFDYAKAYADHLIDDNGNLYFARDELDAIQAGLILFTLYEETKDERYVIAAKKLRSLYDTLNRTSEGGFWHKDKYPYQMWLDGLYMGGPFALKYANMKQEEELFDQVVLQEKLMRKHTKDEKTGLFYHAWDESKKQPWADPETGCSPEFWARSIGWYVMALADMIEELPENHPNREVWTKTLQEMIDAICRYQDEETGLWYQIVDKGDRPDNWLESSGSCLFMYAIAKGINMGYLDPSYLEVLLKAYQGLIQHKTETSKDGAFLLSDICVETTVGDYDYYVSRERSTNDLHGAGAFILAMTELEDLFRSSGK

SEQ ID NO: 86 - protein, 373 aa, synthetic construct
MGSMDQSIAVKSPLEYAEKLAQTIMNTYRPEELPPANRWHYHQGVFLCGLLRLWEKTGDKRYFDYAKAYADHLIDDNGNLYFARDELDAIQAGLILFTLYEETKDERYVIAAKKLRSLYDTLNRTSEGGFWHKDKYPYQMWLDGLYMGGPFALKYANMKQEEELFDQVVLQEKLMRKHTKDEKTGLFYHAWDESKKQPWADPETGCSPEFWARSIGWYVMALADMIEELPENHPNREVWTKTLQEMIDAICRYQDEETGLWYQIVDKGDRPDNWLESSGSCLFMYAIAKGINMGYLDPSYLEVLLKAYQGLIQHKTETSKDGAFLLSDICVQTTVGDYDYYVSRERSTNDLHGAGAFILAMTELEDLFRSSGK

SEQ ID NO: 87 - protein, 377 aa, synthetic construct
MWQQAIGDALGITARNLKKFGDRFPHVSDGSNKYVLNDNTAWTDGFWSGILWLCYEYTGDEQYREGAVRTVASFRERLDRFENLDHHDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYYYLEALLRLERGVTGYWYERGR

SEQ ID NO: 88 - protein, 377 aa, synthetic construct
MWQQAIGDALGITARNLKKFGDRFPHVSDGSNKYVLNDNTCWTDGFWSGILWLCYEYTGDEQYREGAVRTVASFRERLDRFENLDHHDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYYYLEALLRLERGVTGYWYERGR

SEQ ID NO: 89 - protein, 377 aa, synthetic construct
MWQQAIGDALGITARNLKKFGDRFPHVSDGSNKYVLNDNTEWTDGFWSGILWLCYEYTGDEQYREGAVRTVASFRERLDRFENLDHHDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYYYLEALLRLERGVTGYWYERGR

SEQ ID NO: 90 - protein, 377 aa, synthetic construct
MWQQAIGDALGITARNLKKFGDRFPHVSDGSNKYVLNDNTNWTDGFWSGILWLCYEYTGDEQYREGAVRTVASFRERLDRFENLDHHDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYYYLEALLRLERGVTGYWYERGR

SEQ ID NO: 91 - protein, 377 aa, synthetic construct
MWQQAIGDALGITARNLKKFGDRFPHVSDGSNKYVLNDNTSWTDGFWSGILWLCYEYTGDEQYREGAVRTVASFRERLDRFENLDHHDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYYYLEALLRLERGVTGYWYERGR

SEQ ID NO: 92 - protein, 377 aa, synthetic construct
MWQQAIGDALGITARNLKKFGDRFPHVSDGSNKYVLNDNTTWTDGFWSGILWLCYEYTGDEQYREGAVRTVASFRERLDRFENLDHHDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYYYLEALLRLERGVTGYWYERGR

SEQ ID NO: 93 - protein, 377 aa, synthetic construct
MWQQAIGDALGITARNLKKFGDRFPHVSDGSNKYVLNDNTDWTDGFWSGILWLCYEYTGDEQYREGAVRTVASFRERLDRFENLDHHDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYAVRGGISPDDYTIWGDYYYLEALLRLERGVTGYWYERGR

SEQ ID NO: 94 - protein, 377 aa, synthetic construct
MWQQAIGDALGITARNLKKFGDRFPHVSDGSNKYVLNDNTDWTDGFWSGILWLCYEYTGDEQYREGAVRTVASFRERLDRFENLDHHDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYNVRGGISPDDYTIWGDYYYLEALLRLERGVTGYWYERGR

SEQ ID NO: 95 - protein, 377 aa, synthetic construct
MWQQAIGDALGITARNLKKFGDRFPHVSDGSNKYVLNDNTDWTDGFWSGILWLCYEYTGDEQYREGAVRTVASFRERLDRFENLDHADIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYYYLEALLRLERGVTGYWYERGR

SEQ ID NO: 96 - protein, 377 aa, synthetic construct
MWQQAIGDALGITARNLKKFGDRFPHVSDGSNKYVLNDNTDWTDGFWSGILWLCYEYTGDEQYREGAVRTVASFRERLDRFENLDHCDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYYYLEALLRLERGVTGYWYERGR

SEQ ID NO: 97 - protein, 377 aa, synthetic construct
MWQQAIGDALGITARNLKKFGDRFPHVSDGSNKYVLNDNTDWTDGFWSGILWLCYEYTGDEQYREGAVRTVASFRERLDRFENLDHEDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYYYLEALLRLERGVTGYWYERGR

SEQ ID NO: 98 - protein, 377 aa, synthetic construct
MWQQAIGDALGITARNLKKFGDRFPHVSDGSNKYVLNDNTDWTDGFWSGILWLCYEYTGDEQYREGAVRTVASFRERLDRFENLDHGDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYYYLEALLRLERGVTGYWYERGR

SEQ ID NO: 99 - protein, 377 aa, synthetic construct
MWQQAIGDALGITARNLKKFGDRFPHVSDGSNKYVLNDNTDWTDGFWSGILWLCYEYTGDEQYREGAVRTVASFRERLDRFENLDHQDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYYYLEALLRLERGVTGYWYERGR

SEQ ID NO: 100 - protein, 377 aa, synthetic construct
MWQQAIGDALGITARNLKKFGDRFPHVSDGSNKYVLNDNTDWTDGFWSGILWLCYEYTGDEQYREGAVRTVASFRERLDRFENLDHRDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYYYLEALLRLERGVTGYWYERGR

SEQ ID NO: 101 - protein, 377 aa, synthetic construct
MWQQAIGDALGITARNLKKFGDRFPHVSDGSNKYVLNDNTDWTDGFWSGILWLCYEYTGDEQYREGAVRTVASFRERLDRFENLDHSDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYYYLEALLRLERGVTGYWYERGR

SEQ ID NO: 102 - protein, 377 aa, synthetic construct
MWQQAIGDALGITARNLKKFGDRFPHVSDGSNKYVLNDNTDWTDGFWSGILWLCYEYTGDEQYREGAVRTVASFRERLDRFENLDHHDIGFLYSLSAKAQWIVEKDESARKLALDAADVLMRRWRADAGIIQAWGPKGDPENGGRIIIDCLMNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYYYLEALLRLERGVTGYWYERGR

SEQ ID NO: 103 - protein, 377 aa, synthetic construct
MWQQAIGDALGITARNLKKFGDRFPHVSDGSNKYVLNDNTDWTDGFWSGILWLCYEYTGDEQYREGAVRTVASFRERLDRFENLDHHDIGFLYSLSAKAQWIVEKDESARKLALDAADVLMRRWRADAGIIQAWGPKGDPENGGRIIIDCLNNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYYYLEALLRLERGVTGYWYERGR

SEQ ID NO: 104 - protein, 377 aa, synthetic construct
MWQQAIGDALGITARNLKKFGDRFPHVSDGSNKYVLNDNTDWTDGFWSGILWLCYEYTGDEQYREGAVRTVASFRERLDRFENLDHHDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGAYHVRGGISPDDYTIWGDYYYLEALLRLERGVTGYWYERGR

SEQ ID NO: 105 - protein, 377 aa, synthetic construct
MWQQAIGDALGITARNLKKFGDRFPHVSDGSNKYVLNDNTDWTDGFWSGILWLCYEYTGDEQYREGAVRTVASFRERLDRFENLDHHDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAYGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYYYLEALLRLERGVTGYWYERGR

SEQ ID NO: 106 - protein, 377 aa, synthetic construct
MWQQAIGDALGITARNLKKFGDRFPHVSDGSNKYVLNDNTDWTDGFWSGILWLCYEYTGDEQYREGAVRTVASFRERLDRFENLDHHDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIFGDYYYLEALLRLERGVTGYWYERGR

SEQ ID NO: 107 - protein, 377 aa, synthetic construct
MWQQAIGDALGITARNLKKFGDRFPHVSDGSNKYVLNDNTDWTDGFWSGILWLCYEYTGDEQYREGAVRTVASFRERLDRFENLDHHDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSVHVRGGISPDDYTIWGDYYYLEALLRLERGVTGYWYERGR

SEQ ID NO: 108 - protein, 377 aa, synthetic construct
MWQQAIGDALGITARNLKKFGDRFPHVSDGSNKYVLNDNTDWTDGFWSGILWLCYEYTGDEQYREGAVRTVASFRERLDRFENLDHHDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYAYLEALLRLERGVTGYWYERGR

SEQ ID NO: 109 - protein, 377 aa, synthetic construct
MWQQAIGDALGITARNLKKFGDRFPHVSDGSNKYVLNDNTDWTDGFWSGILWLCYEYTGDEQYREGAVRTVASFRERLDRFENLDHHDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYCYLEALLRLERGVTGYWYERGR

SEQ ID NO: 110 - protein, 377 aa, synthetic construct
MWQQAIGDALGITARNLKKFGDRFPHVSDGSNKYVLNDNTDWTDGFWSGILWLCYEYTGDEQYREGAVRTVASFRERLDRFENLDHHDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYFYLEALLRLERGVTGYWYERGR

SEQ ID NO: 111 - protein, 377 aa, synthetic construct
MWQQAIGDALGITARNLKKFGDRFPHVSDGSNKYVLNDNTDWTDGFWSGILWLCYEYTGDEQYREGAVRTVASFRERLDRFENLDHHDIGFLYSLSAKAQWIVEKDESARKLALDAADVLMRRWRADAGIIQAWGPKGDPENGGRIIIDCLLNLPLLLWAGEQTGDPEYRRVAEAHALKSRRFLVRGDDSSYHTFYFDPENGNAIRGGTHQGNTDGSTWTRGQAWGIYGFALNSRYLGNADLLETAKRMARHFLARVPEDGVVYWDFEVPQEPSSYRDSSASAITACGLLEIASQLDESDPERQRFIDAAKTTVTALRDGYAERDDGEAEGFIRRGSYHVRGGISPDDYTIWGDYHYLEALLRLERGVTGYWYERGR

SEQ ID NO: 112 - protein, 377 aa, synthetic construct
MWQQAIGDAVGITRRNLDKFGDRFPHVSNGSNKYVLNDNTDWTDGFWSGILWLCYEYTGDEQYREGAVRTVASFRERLDRFENLDHHDIGFLYSLSSKAQWIVEKDESARQLTLDAADVLMKRWRADAGIIQAWGPGGDPENGGRIIIDCLLNLPLLYWAYEQTGDPEYREVAEAQAEKSRRYLVRGDDSSYHTFYFDPETGEAIRGGTHQGNTDGSTWTRGQAWGIYGFALNYRYTGNPDLLETAKRMARHFLAHLPEDGVAYWDFDAPVTPSTYRDSSASAIVACGLLELASHLDESDPERQRFIDAAKTTVTALRDGYAERDDGEAEGFIRRGSYHVRGGISPDDYMIWGDYYYLEALMRLERGITGYWYERGR

SEQ ID NO: 113 - protein, 377 aa, synthetic construct
MWQQAIGDAVGITRRNLDKFGDRFPHVSNGSNKYVLNDNTDWTDGFWSGILWLCYEYSGDEQYREGAVRTVASFRERLDRFENLDHHDIGFLYSLSAKAQWIVEKDESARQLTLDAADVLMKRWREDGGYIQAWGPKGDPENGGRIIIDCLMNLPLLLWAGEQTGDPEYRRVAEAHALKSRRFLVRGDDSSYHTFYFDPETGEAIRGGTHQGNTDGSTWTRGQAWGIYGFALNYRYTGNPDLLETSKRLARYFLAHLPEDGVAYWDFDAPVTPSTYRDSSASAITAAGLLELASHLDESDPDRQRFIDAAKTTVTALRDGYAERDDGEAEGLLRRGSYHVRGGISPDDYMIWGDYYYLEALLRLERGVTGYWYERGR

SEQ ID NO: 114 - protein, 377 aa, synthetic construct; identical to SEQ ID NO: 112

SEQ ID NO: 115 - protein, 377 aa, synthetic construct
MWQQAIEDALGKTARNIKRFGDRFPHVSDGSNKYVLNDNTDWTDGFWSGILWLCYEYSGDEQYREAAVRTVASFRERLDRKENLDHHDIGFLYSLSAKAQWIVEKDESARKLALDAADVLMRRWRADAGIIQAWGPEGDPENGGRIIIDCLLNLPLLYWAYEQTGDPEYREVAEAHALKSRRFLVRGDDSSYHTFYFDPETGEAIRGGTHQGYTDGSTWTRGQAWGIYGFALNSRYLGNADLLETAKRMARHFLARVPEDGVVYWDFDAPVTPSTYRDSSASAITACGLLEIASQLDESDPERARLIDAAKTTVTALRDGYSERDDGEAEGFIRRGSYHVRGGISPDDYMIWGDYYYLEALLRLERGVTGYWYERGR

SEQ ID NO: 116 - protein, 377 aa, synthetic construct
MWQQAIGDALGITARNIKRFGDRFPHVSNGSNKYVLNDNTDWTDGFWSGILWLCYEYTGDEQFREAAVRTVASFRERLDRFENLDHHDIGFLYSLSAKAQWIVEKDESARKLALDAADVLMRRWRADAGIIQAWGPKGDPENGGRIIIDCLMNLPLLLWAGEQTGDPEYREVAEAQAEKSRRFLVRGDDSSYHTFYFDPETGEAIRGGTHQGYTDGSTWTRGQAWGIYGFALNYRYTGNPDLLETAKRMARHFLARVPEDGVVYWDFEVPQEPSSYRDSSASAIVACGLLEIASQLDESDPDRQRFEDALKTSMTSLRDGYSTRGDGEAEGFIRRGSYHVRGGISPDDYTIWGDYYYLEALLRLERGVTGYWYERGR